(19) United States
(12) Patent Application Publication
(10) Pub. No.: US 2019/0201526 A1
(43) Pub. Date: Jul. 4, 2019

(54) Methods and Compositions for Treating Cancers by Inhibiting Estrogen Signaling in Myeloid-Derived Suppressor Cells

(71) Applicant: The Wistar Institute of Anatomy and Biology, Philadelphia, PA (US)

(72) Inventors: Jose R. Conejo-Garcia, Tampa, FL (US); Nikolaos A. Svoronos, Philadelphia, PA (US)

(21) Appl. No.: 16/331,197

(22) PCT Filed: Sep. 7, 2017
(86) PCT No.: PCT/US17/50500

§ 371 Date: Mar. 7, 2019

Related U.S. Application Data
(60) Provisional application No. 62/384,563, filed on Sep. 7, 2016.

(51) Int. Cl. A61K 39/395; A61K 31/4245; A61K 31/405; C07K 16/28; A61K 45/06; A61P 35/04; G01N 33/74

(52) U.S. Cl. CPC A61K 39/3955; A61K 31/4245; A61K 31/405; C07K 16/2818; G01N 2800/52; A61P 35/04; G01N 33/743; G01N 2333/723; A61K 45/06

(57) ABSTRACT

Compositions and methods are provided for treating an estrogen receptor negative cancer in a subject with an elevated population of estrogen receptor positive myeloid-derived suppressor cells (MDSCs), including administering a therapeutically effective amount of an estrogen receptor antagonist to the subject in need thereof.

### CROSS-REFERENCE TO RELATED APPLICATIONS

This application claims the benefit of U.S. Provisional Application No. 62/384,563, filed Sep. 7, 2016, the entirety of which is incorporated herein by reference.

### GOVERNMENT SUPPORT

This invention was made with government support under Project Nos. R01CA157664, R01CA124515, R01CA178687, and P30CA10815 awarded by the National Institutes of Health. The government has certain rights in the invention.

### FIELD OF THE INVENTION

Therapeutic treatments of cancers, in particular estrogen receptor negative cancers, using methods and compositions to inhibit estrogen signaling in myeloid-derived suppressor cells (MDSCs), are disclosed.

### BACKGROUND OF THE INVENTION

Estrogen and estrogen receptors play a number of roles in tumorogenesis and cancer malignancy. Moreover, while some tumors may represent as estrogen receptor negative, estrogen may yet have an impact on the tumor microenvironment.

The invention provides the unexpected finding that estrogen signaling accelerates the progression of different estrogen insensitive tumors by contributing to deregulated myelopoiesis by both driving the mobilization of myeloid-derived suppressor cells (MDSCs) and enhancing their intrinsic immunosuppressive activity. Accordingly, the methods and compositions of the invention are provided for inhibiting estrogen receptor signaling in MDSCs for the treatment of estrogen insensitive cancers.

### SUMMARY OF THE INVENTION

Ablating estrogenic activity delays the progression of multiple tumors independently of the tumor cell responsiveness, owing to a decrease in the mobilization and immunosuppressive activity of MDSCs, which boosts T-cell-dependent anti-tumor immunity. Conversely, enhanced estrogenic activity (e.g., as in BRCA1-mutation carriers) contributes to accelerated malignant progression. The invention provides a mechanistic rationale to block estrogen signaling with estrogen receptor antagonists to boost the effectiveness of novel anti-cancer immunotherapies by inhibiting the mobilization and activity of tumor-induced MDSCs.

The invention described herein includes methods and compostions for treating estrogen receptor negative cancer in a subject with estrogen receptor positive myeloid-derived suppressor cells (MDSCs). The invention described herein also includes kits for practicing such methods. In some embodiments, the subject has an elevated population of estrogen receptor positive MDSCs.

In some embodiments, the invention includes a method of treating estrogen receptor negative cancer in a subject with an elevated population of estrogen receptor positive MDSCs, the method including the step of administering a therapeutically effective amount of one or more estrogen receptor antagonists to the subject in need thereof.

In some embodiments, the invention includes a method of treating estrogen receptor negative cancer in a subject that includes the steps of: (a) obtaining a blood sample from the subject; (b) analyzing the blood sample for MDSCs and testing the MDSCs with an estrogen receptor-specific protein assay; (c) determining whether the MDSCs are estrogen receptor positive; and (d) administering a therapeutically effective amount of one or more estrogen receptor antagonists to the subject in need thereof.

In certain embodiments, the methods of the invention may include the administration of a therapeutically effective amount of an immunotherapeutic agent.

In some embodiments, the invention includes a pharmaceutical composition for treating estrogen receptor negative cancer in a subject with an elevated population of estrogen receptor positive MDSCs, where the composition includes one or more estrogen receptor antagonists and an immunotherapeutic agent in therapeutically effective amounts, and a pharmaceutically acceptable carrier.

In some embodiments of the foregoing methods and pharmaceutical compositions, the immunotherapeutic agent may include one or more of a CTLA-4 inhibitor, a PD-1 inhibitor, a PD-L1 inhibitor, and an IDO inhibitor.

In some embodiments of the methods and pharmaceutical compositions described herein, the estrogen receptor negative cancer may be selected from the group consisting of lung cancer, breast cancer, endometrial cancer, skin cancer, ovarian cancer, gastric cancer, colorectal cancer, brain cancer, renal cancer, bladder cancer, ureter cancer, pancreatic cancer, prostate cancer, thyroid cancer, head and neck cancer, liver cancer, lymphoid cancer, and splenic cancer.

In some embodiments of the foregoing methods and pharmaceutical compositions, the estrogen receptor negative cancer may be selected from the group consisting of lung cancer, breast cancer, endometrial cancer, skin cancer (e.g., melanoma), and ovarian cancer.

In some embodiments of the foregoing methods and pharmaceutical compositions, the one or more estrogen receptor antagonists may be selected from the group consisting of: methylpiperidino pyrazole (MPP), THIQ-40, GDC-0927, H3B-6545, VP-128, (E)-3-(3,5-difluoro-4-((1R,3R)-2-(2-fluoro-2-methyl-propyl)-3-methyl-2,3,4,9-tetrahydro-1H-pyrido[3,4-b]indol-1-yl)phenyl)acrylic acid (AZD9496), (11β,17β)-11-[4-[[5-[(4,4,5,5,5-Pentafluoropentyl)sulfonyl]pentyl]oxy]phenylestra-1,3,5,(10)-triene-3,17-diol (RU 58668), 13-methyl-7-[9-(4,4,5,5,5-pentafluoropentylsulfinyl)nonyl]-7,8,9,11,12,13,14,15,16,17-decahydro-6H-cyclopenta[a]-phenanthrene-3,17-diol (fulvestrant), N-butyl-11-[(7R,8S,9S,13S,14S,17S)-3,17-dihydroxy-13-methyl-6,7,8,9,11,12,14,15,16,17-decahydrocyclopena[a]phenanthren-7-yl]-N-methyl-undecanamide (ICI 164384), (+)-7-pivaloyloxy-3-(4′-pivaloyloxyphenyl)-4-methyl-2-(4″-(2″-piperidinoethoxy)phenyl)-2H-benzopyran (EM-800), and (2S)-3-(4-hydroxyphenyl)-4-methyl-2-[4-[2-(1-piperidyl)ethoxy]phenyl]-2H-chromen-7-ol (EM-652).

### BRIEF DESCRIPTION OF THE DRAWINGS

The foregoing summary, as well as the following detailed description of the invention, will be better understood when read in conjunction with the appended drawings.

FIGS. 1A to 1F illustrate that estrogen-depletion impairment of ovarian tumor progression is independent of tumor cell signaling and is immune dependent. Frozen human ovarian tumor sections stained for ERα (FIG. 1A). Scale bar indicates 1 μm. Reverse transcription qPCR for ESR1 expression in myeloid (CD45+CD11b+) and non-myeloid (CD45+CD11b−) cells isolated from dissociated ovarian tumor or bone marrow from cancer patients (NTC indicates no template control) (FIG. 1B). Western-blot for ERα (66 kDa) expression by ID8-Defb29/Vegf-α tumor cells and myeloid derived suppressor cells isolated from mouse tumors (FIG. 1C). Proliferation relative to vehicle of ID8-Defb29/Vegf-α and MCF-7 (positive control) cells in response increasing doses of estradiol (in steroid-free media) and fulvestrant as determined by MTS assay (FIG. 1D). Survival of WT oophorectomized (OVX) or sham-operated mice challenged with intraperitoneal ID8-Defb29/Vegf-α (FIG. 1E). Survival of OVX or sham-operated Rag1 KO mice challenged with i.p. ID8-Defb29/Vegf-α (FIG. 1F). Representative survival curves are shown for multiple independent experiments. *p<0.05.

FIGS. 2A to 2C illustrate that estrogen suppresses anti-tumor T-cell responses. Proportion of CD45+ cells isolated from ID8-Defb29/Vegf-α peritoneal tumors that are T cells (CD45+CD3+γ δ-TCR−) (FIG. 2A). Proportion of activated CD44+CD69+ double positive cells among CD4+ and CD8+ T cells (FIG. 2B). ELISpot analysis of T cells isolated from ID8-Defb29/Vegf-α peritoneal tumors stimulated with tumor lysate-pulsed BM-derived dendritic cells. Results shown are representative of multiple independent experiments (FIG. 2C). *p<0.05.

FIGS. 3A to 3F illustrate that estrogen drives accumulation of myelomonocytic (M-MDCS) and granulocytic (G-MDSC) myeloid-derived suppressor cells and increases the immunosuppressive potential of G-MDSCS. LLC lung tumor progression in WT mice treated with vehicle (Vh), estradiol (E2), or oophorectomy (OVX) challenged intraperitoneal (left) or subcutaneous (right) (FIG. 3A). Expression and quantification of M-MDSCs (Ly6ChighLy6G−) and G-MDSCs (Ly6C+Ly6G+) in the spleen of ID8-Defb29/Vegf-α peritoneal tumor-bearing mice (FIGS. 3B and 3C). Expression and quantification of M-MDSCs (Ly6ChighLy6G−) and G-MDSCs (Ly6C+Ly6G+) in the peritoneal cavity of ID8-Defb29/Vegf-α peritoneal tumor-bearing mice (FIGS. 3D and 3E). Dilution of Cell Trace Violet in labeled T cells activated with anti-CD3/CD28 beads co-cultured with varying ratios of M- and G-MDSCs isolated from ID8-Defb29/Vegf-α tumors (FIG. 3F). *p<0.05.

FIGS. 4A to 4E illustrate that host estrogen receptor α (ERα) activity is required for E2-driven tumor acceleration and optimal MDSC accumulation. Survival of WT and ERα KO mice treated with Vh or E2 and challenged with i.p. ID8-Defb29/Vegf-α tumors (FIG. 4A). Survival of WT mice lethally irradiated (10 Gy) and reconstituted with WT or ERα KO bone marrow treated with Vh or E2 and challenged with i.p. ID8-Defb29/Vegf-α tumors (FIG. 4B). Representative survival curves shown for multiple independent experiments. Expression and quantification of WT and ERα KO MDSCs (CD45+CD11b+Gr-1+) in the spleens of tumor bearing mice lethally irradiated and reconstituted with a 1:1 mix of WT CD45.1+ and ERα KO CD45.2+ bone marrow (FIGS. 4C and 4D). Expression of Ly6C and Ly6G by WT and ERα KO CD11b+MHC-II-cells in the spleens of mixed BM reconstituted mice (FIG. 4E). *p<0.05.

FIGS. 5A to 5E illustrate that optimal MDSC expansion and suppressive activity is dependent on estrogen signaling. Expression of Ly6C and Ly6G (left) or MHC-II and CD11c (right) by naïve mouse WT bone marrow cultured with GM-CSF+IL6 and treated with Vh or 2 anti-estrogen MPP for 3 and 6 days (FIG. 5A). Total number of M-MDSCs and G-MDSCs after culturing naïve WT mouse BM with GM-CSF+IL6 and treating with 2 μM MPP for 6 days (FIG. 5B). Dilution of Cell Trace Violet by labeled T cells activated with anti-CD3/CD28 beads co-cultured with varying ratios of G- or M-MDSCs isolated from 6-day bone marrow cultures treated with Vh, 100 ng/mL E2, or 2 μM MPP (FIG. 5C). Expansion of human M-MDSCs (CD45+HLA-DR-CD11b+CD33+CD14+) and G-MDSCs (CD45+HLA-DR-CD11b+CD33+CD15+) from lung cancer patient bone marrow cultured in GM-CSF+IL6 and treated with Vh, 2 or 10 μM MPP (FIG. 5D). Total number of human M- and G-MDSCs derived from lung cancer patient bone marrow (FIG. 5E). *p<0.05.

FIGS. 6A to 6E illustrate that estrogen increases cytokine-induced STAT3 during MDSC expansion. Phosphorylated and total STAT3 protein expression in M- and G-MDSCs isolated from the peritoneal cavity and spleens of i.p. tumor-bearing oophorectomized or E2-treated mice (FIG. 6A). pSTAT3 and total STAT3 protein expression in in vitro BM-derived MDSC cultures treated with Vh, 100 ng/mL E2, or 2 μM MPP (FIG. 6B). RNA expression of STAT3 in in vitro BM-derived MDSC cultures treated with Vh, E2, or MPP (FIG. 6C). In vitro BM-derived MDSC surface expression of IL6Rα and GP-130 in response to Vh, E2, or MPP treatment (FIG. 6D). Jak2 RNA (left) and Jak2 protein (right) expression of in vitro BM-derived MDSC in response to Vh, E2, or MPP treatment (FIG. 6E). *p<0.05.

FIGS. 7A to 7C illustrate that T cell-intrinsic ERα activity suppresses anti-tumor response, but is insufficient to abrogate the effectiveness of tumor-primed T cells. Intratumoral T cell expression of CD44 and CD69 in mice lethally irradiated and reconstituted with a 1:1 mix of WT CD45.1+ and ERα KO CD45.2+ bone marrow (FIG. 7A). ELISpot analysis of intratumoral WT and ERα KO T cells FACS-isolated from tumor-bearing mice stimulated with tumor antigen loaded BMDCs (FIG. 7B). Survival of tumor-bearing Vh or E2 treated mice following adoptive transfer of tumor antigenprimed WT or ERα KO T cells. Representative survival curves shown for multiple independent experiments (FIG. 7C). *p<0.05.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present is also encompassed in the methods of the invention.

As used herein, cells are considered to be “receptor-positive,” if they show distinct nuclear staining with an adequate receptor-specific staining method, e.g., with a receptor specific antibody for an estrogen receptor (as described herein), which is either labelled as such, or is detected by a secondary antibody, which is labelled. For example, cells (which may be isolated from a subject's tissue, blood, or serum) such as myeloid-derived suppressor cells (MDSCs) may be estrogen receptor positive (ER (+)).

As used herein, cells are considered to be “receptor-negative,” if they do not show distinct nuclear staining with an adequate receptor-specific staining method, e.g., with a receptor specific antibody for an estrogen receptor (as described herein) which is either labelled as such, or is detected by a secondary antibody, which is labelled. For example, cells (which may be isolated from a subject's tissue, blood, or serum) such as lung cancer (e.g., non-small cell lung or bronchoalveoloar carcinoma) cells, breast cancer (e.g., mammary carcinoma) cells, endometrial cancer cells, skin cancer (e.g., melanoma) cells, colorectal (e.g., colon and/or rectal) cancer cells, gastric cancer (e.g., gastrointestinal tumor) cells, brain cancer (e.g., glioblastoma) cells, bladder/ureter cancer (e.g., urothelial carcinoma) cells, renal cancer cells, pancreatic cancer (e.g., pancreatic adenocarcinoma) cells, prostate cancer cells, thyroid cancer (e.g., anaplastic thyroid carcinoma) cells, head and neck cancer (e.g., tongue squamous cell carcinoma or head and neck squamous cell carcinoma), liver cancer cells, lymphoid/splenic cancer cells, or ovarian cancer cells, may be estrogen receptor negative (ER (−)).

The terms “active pharmaceutical ingredient” and “drug” refers to any compound that is biologically active, including individual drugs in the pharmaceutical compositions described herein, such as any estrogen receptor antagonists or immunotherapeutic agents. As used herein, “immunotherapeutic agents” may include one or more of a CTLA-4 inhibitor, a PD-1 inhibitor, a PD-L1 inhibitor, an IDO inhibitor, an IL-6 inhibitor, and an IL-6R inhibitor. In some embodiments, “immunotherapeutic agents” may encompass small-molecule inhibitors or antibodies, including monoclonal antibodies (e.g., anti-PD-1 antibodies).

The term “elevated population,” either as stated or in conjunction with the elevated population of cells, refers to a number of cells (e.g., ER (+) MDSCs) found in a subject's bodily fluid (e.g., blood) or tissue that is measurably greater than the population of the same cells that is observed in a normal subject's bodily fluid or tissue.

The term “reduced population,” either as stated or in conjunction with the reduced population of cells, refers to a number of cells (e.g., ER (+) MDSCs) found in a subject's bodily fluid (e.g., blood) or tissue that is measurably less than the population of the same cells that is observed in a normal subject's bodily fluid or tissue.

The term “elevated concentration,” either as stated or in conjunction with the elevated concentration of a protein, antibody, or other relevant biomolecule (e.g., “elevated IgE concentration”), refers to a concentration of a protein, antibody, or other relevant biomolecule found in a subject's bodily fluid (e.g., plasma) that is measurably greater than the concentration of the same protein, antibody, or other relevant biomolecule that is observed in a normal subject's bodily fluid.

The term “reduced concentration,” either as stated or in conjunction with the reduced concentration of a protein, antibody, or other relevant biomolecule (e.g., “reduced IgE concentration”), refers to a concentration of a protein, antibody, or other relevant biomolecule found in a subject's bodily fluid (e.g., plasma) that is measurably less than the concentration of the same protein, antibody, or other relevant biomolecule that is observed in a normal subject's bodily fluid.

The term “subject” refers to a mammal such as a mouse or a human. In certain embodiments described herein, the subject is a human subject or human patient.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit in a subject. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “estrogen receptor antagonists” or “ER antagonists” refers to an agent that binds to an estrogen receptor and subsequently decreases the activity of the estrogen receptor and thereby prevents estrogens from expressing their effects on estrogen dependent target tissues, consequently antagonizing a variety of estrogen-dependent processes. The ER antagonists described herein are “pure” ER antagonists and do not have partial estrogenic action as in the case of the selective estrogen receptor modulators (SERMs) which exhibit ER antagonistic properties in some tissues and estrogenic properties in others (e.g., tamoxifen).

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier,” “pharmaceutically acceptable excipient,” or “excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, from 0% to 10%, from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or enantiomers can be prepared by asymmetric syntheses. See, for example, Jacques et al., Enantiomers, Racemates and Resolutions, Wiley Interscience, New York (1981); Eliel, Stereochemistry of Carbon Compounds, McGraw-Hill, New York (1962); and Eliel and Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

“Solvate” refers to a compound in physical association with one or more molecules of a pharmaceutically acceptable solvent.

Compounds used in the methods or compositions of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

The active pharmaceutical ingredients and/or drugs described herein also include antibodies. The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to, e.g., PD-1, can be made using knowledge and skill in the art of injecting test subjects with PD-1 antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion”), as used herein, refers to one or more fragments of an antibody that retain the ability to specifically bind to an antigen (e.g., PD-1 antigen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird et al., Science 1988, 242, 423-426; and Huston et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to a therapeutic moiety, such as a bacterial toxin, a cytotoxic drug or a radionuclide-containing toxin. Toxic moieties can be conjugated to antibodies of the invention using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 1986, 321, 522-525; Riechmann et al., Nature 1988, 332, 323-329; and Presta, Curr. Op. Struct. Biol. 1992, 2, 593-596.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki et al. Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino et al., Biochem. 1975, 14, 5516-5523.

The term “conservative amino acid substitutions” means amino acid sequence modifications which do not abrogate the binding of the antibody to the antigen. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-HER2 antibody is preferably replaced with another amino acid residue from the same class. Methods of identifying amino acid conservative substitutions which do not eliminate antigen binding are well-known in the art (see, e.g., Brummell et al., Biochemistry 1993, 32, 1180-1187; Kobayashi et al., Protein Eng. 1999, 12, 879-884 (1999); and Burks et al., Proc. Natl. Acad. Sci. USA 1997, 94, 412-417).

The terms “sequence identity,” “percent identity,” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

Certain embodiments of the invention comprise a variant of an antibody, e.g., an anti-HER2 antibody. As used herein, the term “variant” encompasses but is not limited to antibodies which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody.

The term “radioisotope-labeled complex” refers to both non-covalent and covalent attachment of a radioactive isotope, such as ⁹⁰Y, ¹¹¹In or ¹³¹I, to an antibody, including conjugates.

The term “biosimilar” means a biological product that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference anti-CD20 monoclonal antibody is rituximab, an anti-CD20 biosimilar monoclonal antibody approved by drug regulatory authorities with reference to rituximab is a “biosimilar to” rituximab or is a “biosimilar thereof” of rituximab. In Europe, a similar biological or “biosimilar” medicine is a biological medicine that is similar to another biological medicine that has already been authorized for use by the European Medicines Agency (EMA). The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorised by the EMA. However, in some instances, the biosimilar may be compared to a biological medicinal product which has been authorised outside the European Economic Area (a non-EEA authorised “comparator”) in certain studies. Such studies include for example certain clinical and in vivo non-clinical studies. As used herein, the term “biosimilar” also relates to a biological medicinal product which has been or may be compared to a non-EEA authorised comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorised or considered suitable for authorisation. In certain circumstances, the biosimilar exhibits different binding characteristics as compared to the reference medicinal product, wherein the different binding characteristics are considered by a Regulatory Authority such as the EMA not to be a barrier for authorisation as a similar biological product. The term “biosimilar” is also used synonymously by other national and regional regulatory agencies.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, and solid tumors developed from lung cancer (e.g., non-small cell lung or bronchoalveoloar carcinoma), breast cancer (e.g., mammary carcinoma), endometrial cancer, skin cancer (e.g., melanoma), colorectal (e.g., colon and/or rectal) cancer, gastric cancer (e.g., gastrointestinal tumor), brain cancer (e.g., glioblastoma), renal cancer, bladder/ureter cancer (e.g., urothelial carcinoma), pancreatic cancer (e.g., pancreatic adenocarcinoma), prostate cancer, thyroid cancer (e.g., anaplastic thyroid carcinoma), head and neck cancer (e.g., tongue squamous cell carcinoma or head and neck squamous cell carcinoma), liver cancer, lymphoid/splenic cancer, or ovarian cancer. The tissue structure of solid tumors includes interdependent tissue compartments including the parenchyma (cancer cells) and the supporting stromal cells in which the cancer cells are dispersed and which may provide a supporting microenvironment.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

### Myeloid-Derived Suppressor Cells (MDSCs) and Estrogen Signaling

Estrogens are pleiotropic steroid hormones known to influence many biological processes that ultimately affect homeostasis, such as development and metabolism. Estrogens bind to two high-affinity receptors (ERs; α and β) that activate similar but not identical response elements and are differentially expressed in multiple tissues. Due to their pathogenic role in accelerated malignant progression, ER (+) breast cancers have been commonly treated with tamoxifen. Tamoxifen, however, has mixed antagonist/agonist effect on Estrogen Receptors (ERs), depending on the cell type. Correspondingly, alternative interventions are currently evolving as results from clinical testing emerge. In contrast to breast cancer, anti-estrogen therapies have proven to be effective in only some ovarian cancer patients. However, these studies were exclusively focused on ER (+) cancer patients, which only represent 31% for ERα and 60% for ERβ, and therefore did not provide any insight into the effects of estrogen activity on nontumor cells.

Besides tumor cells, the tumor microenvironment plays an important role in determining malignant progression as well as response to various therapies. In particular, it is becoming evident that tumors elicit immune responses that ultimately impact survival. In ovarian cancer, for instance, the presence of tumor-infiltrating lymphocytes is a major positive prognostic indicator of tumor survival, and multiple T-cell inhibitory pathways have been identified.

In addition to tumor cells, both ERs are expressed by most immune cell types, including T-cells, B-cells and NK cells, in which ERα46 is the predominant isoform. Correspondingly, estrogens influence helper CD4 T cell differentiation favoring humoral Th2 over cell-mediated Th1 responses. Women have higher levels of estrogen than men, which may contribute to differences in the incidence of certain autoimmune diseases. Most importantly, various cancers exhibit sex biases that are at least partly explained by hormonal differences. Obesity, which is associated with increased adipocyte production of estrogens, is also a risk factor for a number of cancers. Changes in estrogen levels in women caused by menstruation, menopause, and pregnancy are associated with changes in the immune system, which could ultimately affect disease susceptibility. Despite growing evidence implicating estrogen as a fundamental mediator of inflammation, currently little is known about its potential role in antitumor immune responses, and particularly in patients without direct estrogen signaling on tumor cells but with a strongly responsive immune-environment.

Among suppressors of anti-tumor immune responses, factors driving tumor-associated inflammation universally induce aberrant myelopoiesis in solid tumors, which fuels malignant progression in part by generating immunosuppressive myeloid cell populations. In ovarian cancer, deregulated myelopoiesis results in the mobilization of Myeloid-Derived Suppressor Cells (MDSCs) from the bone marrow and, eventually, the accumulation of tumorpromoting inflammatory Dendritic Cells (DCs) with immunosuppressive activity in solid tumors, while canonical macrophages buildup in tumor ascites. Although all these cell types express at least ERα and are influenced by estrogen signaling, how estrogens impact the orchestration and maintenance of protective anti-tumor immunity remains elusive. As shown herein, estrogens, independently of the sensitivity of tumor cells to estrogen signaling, are an important mechanism underlying pathological myelopoiesis in a number of different cancers, including breast cancer and ovarian cancer. Estrogens drive MDSC mobilization and augment their immunosuppressive activity, which directly facilitates malignant progression. Moreover, the data provided herein describes a mechanistic insight into how augmented estrogenic activity contributes to tumor initiation (e.g., in BRCA1-mutation carriers), and provides a rationale for blocking estrogen signals to boost the effectiveness of anti-cancer immunotherapies.

Accordingly, in some embodiments, the invention includes a method of treating an ER (−) cancer in a subject with an elevated population of estrogen ER (+) MDSCs, which includes administering a therapeutically effective amount of one or more estrogen receptor antagonists, as described herein, to the subject in need thereof.

MDSCs may be found in humans as immature myeloid cells. Therefore, as used herein, the terms myeloid-derived suppressor cells (MDSCs) and immature myeloid cells are understood to be interchangeable.

In some embodiments, the method further includes the step of administering a therapeutically effective amount of one or more immunotherapeutic agents, as described herein.

In some embodiments, and without being limited to any one theory of the invention, the administration of one or more estrogen receptor antagonists may inhibit estrogen signaling by ER (+) MDSCs and thereby increase the effectiveness of co-administered immunotherapeutic agents.

### Estrogen Receptor Antagonists

In some embodiments, the methods and/or compositions described herein include one or more ER antagonists. In certain embodiments, the ER antagonists of the invention include, without limitation, one or more of: methylpiperidino pyrazole ((MPP), as described in Sun, et al. Endocrinol. (2002) 143: 941-947); THIQ-40 (as described in Burks, et al. J. Med. Chem. (2017) 60: 2790-2818); GDC-0927 (as provided by Genentech, Inc.); H3B-6545 (as provided by H3 Biomedicine, Inc.); VP-128 (as described in Themsche, et al. Endocrine-Related Cancer (2009) 16: 1185-1195); (E)-3-(3,5-difluoro-4-((1R,3R)-2-(2-fluoro-2-methyl-propyl)-3-methyl-2,3,4,9-tetrahydro-1H-pyrido[3,4-b]indol-1-yl)phenyl)acrylic acid (AZD9496, as described in Weir, et al. Cancer Res (2016) 76:3307-3318); (11β,17β)-11-[4-[[5-[(4,4,5,5,5-Pentafluoropentyl)sulfonyl]pentyl]oxy]phenylestra-1,3,5,(10)-triene-3,17-diol (RU 58,668), described in Van de Velde et al, Ann NY Acad. Sci., 761: 164-175 (1995); 13-methyl-7-[9-(4,4,5,5,5-pentafluoropentylsulfinyl)nonyl]-7,8,9,11,12,13,14,15,16,17-decahydro-6H-cyclopenta[a]-phenanthrene-3,17-diol (ICI 182,780 or fulvestrant) as described in EP 0138504; N-butyl-11-[(7R,8S,9S,13S,14S,17S)-3,17-dihydroxy-13-methyl-6,7,8,9,11,12,14,15,16,17-decahydrocyclopena[a]phenanthren-7-yl]-N-methyl-undecanamide (ICI 164,384), described in Wakeling and Bowler, J. Endocrin., 112:R7-R110 (1987); (+)-7-pivaloyloxy-3-(4′pivaloyloxyphenyl)-4-methyl-2-(4″(2″piperidinoethoxy)phenyl)-2H-benzopyran (EM-800 or SCH 57050) as described in WO 96/26201; and (2S)-3-(4-hydroxyphenyl)-4-methyl-2-[4-[2-(1-piperidyl)ethoxy]phenyl]-2H-chromen-7-ol (EM-652 or SCH 57068).

In an embodiment, the ER antagonist may be methylpiperidino pyrazole (MPP) (as shown in Formula I), or a pharmaceutically acceptable salt, solvate, hydrate, cocrystal, or prodrug thereof.

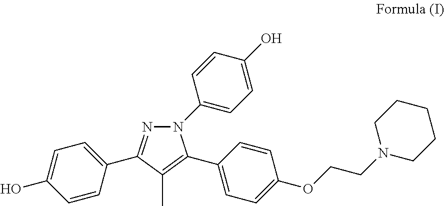 [SMILES: Cc1c(-c2ccc(O)cc2)nn(-c2ccc(O)cc2)c1-c1ccc(OCCN2CCCCC2)cc1]

In an embodiment, the ER antagonist may be THIQ-40 (as shown in Formula II), or a pharmaceutically acceptable salt, solvate, hydrate, cocrystal, or prodrug thereof.

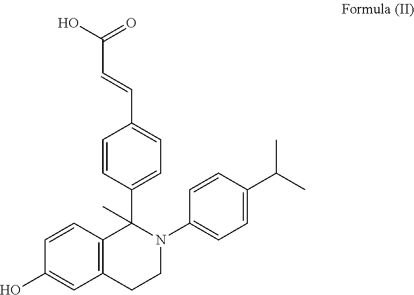 [SMILES: CC(C)c1ccc(N2CCc3cc(O)ccc3C2(C)c2ccc(/C=C/C(=O)O)cc2)cc1]

In an embodiment, the ER antagonist may be GDC-0927, as provided by Genentech, Inc., or a pharmaceutically acceptable salt, solvate, hydrate, cocrystal, or prodrug thereof.

In an embodiment, the ER antagonist may be H3B-6545, as provided by H3 Biomedicine, Inc., or a pharmaceutically acceptable salt, solvate, hydrate, cocrystal, or prodrug thereof.

In an embodiment, the ER antagonist may be VP-128, a 17β-oestradiol (E₂)-linked platinum (II) hybrid (as shown in Formula III), or a pharmaceutically acceptable salt, solvate, hydrate, cocrystal, or prodrug thereof.

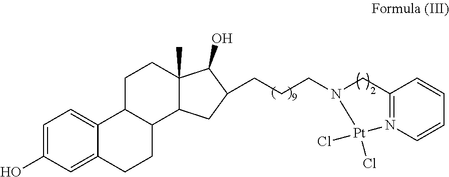

In an embodiment, the ER antagonist may be (E)-3-(3,5-difluoro-4-((1R,3R)-2-(2-fluoro-2-methyl-propyl)-3-methyl-2,3,4,9-tetrahydro-1H-pyrido[3,4-b]indol-1-yl)phenyl)acrylic acid (AZD9496) (as shown in Formula IV), or a pharmaceutically acceptable salt, solvate, hydrate, cocrystal, or prodrug thereof.

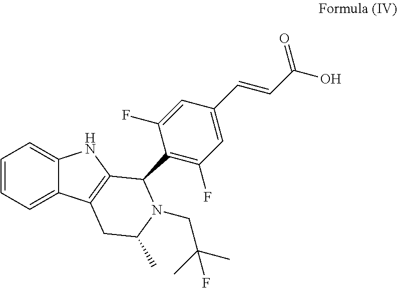 [SMILES: C[C@@H]1Cc2c([nH]c3ccccc23)[C@@H](c2c(F)cc(/C=C/C(=O)O)cc2F)N1CC(C)(C)F]

In an embodiment, the ER antagonist may be (11β,17β)-11-[4-[[5-[(4,4,5,5,5-Pentafluoropentyl)sulfonyl]pentyl]oxy]phenylestra-1,3,5,(10)-triene-3,17-diol (as shown in Formula V), or a pharmaceutically acceptable salt, solvate, hydrate, cocrystal, or prodrug thereof.

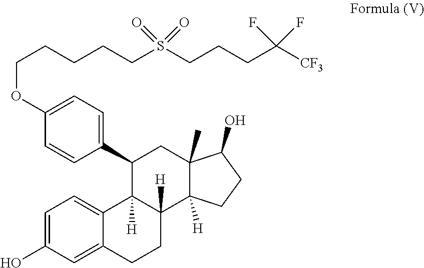

In an embodiment, the ER antagonist may be 13-methyl-7-[9-(4,4,5,5,5-pentafluoropentylsulfinyl)nonyl]-7,8,9,11,12,13,14,15,16,17-decahydro-6H-cyclopenta[a]-phenanthrene-3,17-diol (as shown in Formula VI) or a pharmaceutically acceptable salt, solvate, hydrate, cocrystal, or prodrug thereof.

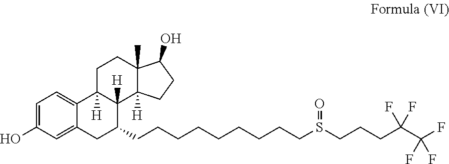

In an embodiment, the ER antagonist may be N-butyl-11-[(7R,8S,9S,13S,14S,17S)-3,17-dihydroxy-13-methyl-6,7,8,9,11,12,14,15,16,17-decahydrocyclopena[a]phenanthren-7-yl]-N-methyl-undecanamide (as shown in Formula VII) or a pharmaceutically acceptable salt, solvate, hydrate, cocrystal, or prodrug thereof.

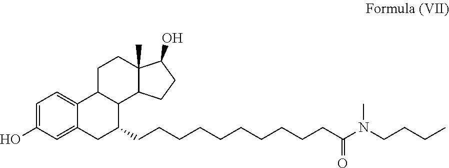 [SMILES: CCCCN(C)C(=O)CCCCCCCCCC[C@@H]1Cc2cc(O)ccc2C2CC[C@@]3(C)C(CC[C@@H]3O)C21]

In an embodiment, the ER antagonist may be (+)-7-pivaloyloxy-3-(4′-pivaloyloxyphenyl)-4-methyl-2-(4″-(2″-piperidinoethoxy)phenyl)-2H-benzopyran (as shown in Formula VIII) or a pharmaceutically acceptable salt, solvate, hydrate, cocrystal, or prodrug thereof.

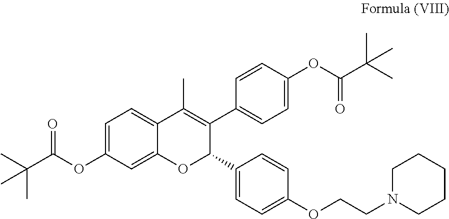 [SMILES: CC1=C(c2ccc(OC(=O)C(C)(C)C)cc2)[C@H](c2ccc(OCCN3CCCCC3)cc2)Oc2cc(OC(=O)C(C)(C)C)ccc21]

In an embodiment, the ER antagonist may be (2S)-3-(4-hydroxyphenyl)-4-methyl-2-[4-[2-(1-piperidyl)ethoxy]phenyl]-2H-chromen-7-ol (as shown in Formula IX) or a pharmaceutically acceptable salt, solvate, hydrate, cocrystal, or prodrug thereof.

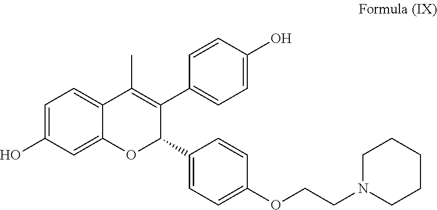 [SMILES: CC1=C(c2ccc(O)cc2)[C@H](c2ccc(OCCN3CCCCC3)cc2)Oc2cc(O)ccc21]

### CTLA-4 Inhibitors

In some embodiments, the methods and/or compositions described herein include a combination of an ER antagonist and one or more immunotherapeutic agents. In some embodiments, the immunotherapeutic agents may include one or more CTLA-4 inhibitors, such as an anti-CTLA-4 antibody.

As used herein, the term “anti-CTLA-4 antibody” may refer to any antibody or protein that binds to CTLA-4 and may include ipilimumab and/or tremulimumab. In an embodiment, the anti-CTLA-4 antibody may be any anti-CTLA-4 antibody known in the art. In particular, it is one of the anti-CTLA-4 antibodies described in more detail in the following paragraphs. In some embodiments, the compositions described herein provide a combination of an anti-CTLA-4 antibody with an ER receptor antagonist, or methods of using a combination of an anti-CTLA-4 antibody with an ER receptor antagonist. In a preferred embodiment, the anti-CTLA-4 antibody is an anti-CTLA-4 monoclonal antibody.

In an embodiment, the anti-CTLA-4 antibody is ipilimumab (trade name YERVOY, also known as MDX-010 and MDX-101), or a fragment, derivative, conjugate, variant, radioisotope-labeled complex, or biosimilar thereof. Ipilimumab is described in U.S. Pat. Nos. 6,984,720; 7,605,238; 8,017,114; 8,318,916; and 8,784,815, the disclosures of which are incorporated by reference herein. Ipilimumab is commercially available from sources including Bristol-Myers Squibb, Inc. The amino acid sequence for the heavy chain of ipilimumab is set forth in SEQ ID NO:1. The amino acid sequence for the light chain of ipilimumab is set forth in SEQ ID NO:2.

In an embodiment, the anti-CTLA-4 monoclonal antibody is an anti-CTLA-4 biosimilar monoclonal antibody approved by drug regulatory authorities with reference to ipilimumab. In an embodiment, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is ipilimumab. In some embodiments, the one or more post-translational modifications are selected from one or more of: glycosylation, oxidation, deamidation, and truncation. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is ipilimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is ipilimumab. In some embodiments, the biosimilar comprises one or more excipients selected from tris-hydrochloride, sodium chloride, mannitol, pentetic acid, polysorbate 80, sodium hydroxide, and hydrochloric acid.

In an embodiment, the anti-CTLA-4 antibody is tremelimumab (also known as ticilimumab and CP-675,206), and fragments, derivatives, conjugates, variants, radioisotope-labeled complexes, and biosimilars thereof. Tremelimumab is described in U.S. Pat. Nos. 6,682,736; 7,109,003; 7,132,281; 7,411,057; 8,143,379; 8,491,895; and/or 8,883,984; the disclosures of which are incorporated by reference herein. Tremelimumab is commercially available from sources including AstraZeneca, Inc. The amino acid sequence for the heavy chain of tremelimumab is set forth in SEQ ID NO:11. The amino acid sequence for the light chain of tremelimumab is set forth in SEQ ID NO:12.

In an embodiment, the anti-CTLA-4 monoclonal antibody is an anti-CTLA-4 biosimilar monoclonal antibody approved by drug regulatory authorities with reference to tremelimumab. In an embodiment, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tremelimumab. In some embodiments, the one or more post-translational modifications are selected from one or more of: glycosylation, oxidation, deamidation, and truncation. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is tremelimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tremelimumab. In some embodiments, the biosimilar comprises one or more excipients selected from tris-hydrochloride, sodium chloride, mannitol, pentetic acid, polysorbate 80, sodium hydroxide, and hydrochloric acid.

In an embodiment, an anti-CTLA-4 antibody selected from the group consisting of ipilimumab and tremelimumab, and/or Fab fragments, antigen-binding fragments, derivatives, conjugates, variants, and radioisotope-labeled complexes thereof, is administered to a subject by infusing a dose selected from the group consisting of about 10 mg, about 20 mg, about 25 mg, about 50 mg, about 75 mg, 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, and about 2000 mg. In an embodiment, the anti-CTLA-4 antibody is administered weekly. In an embodiment, the anti-CTLA-4 antibody is administered every two weeks. In an embodiment, the anti-CTLA-4 antibody is administered every three weeks. In an embodiment, the anti-CTLA-4 antibody is administered monthly. In an embodiment, the anti-CTLA-4 antibody is administered at a lower initial dose, which is escalated when administered at subsequent intervals administered monthly.

In an embodiment, the invention provides a method as described herein comprising co-administering, to a mammal in need thereof, therapeutically effective amounts of an anti-CTLA-4 antibody, or a fragment, derivative, conjugate, variant, radioisotope-labeled complex, or biosimilar thereof, wherein the anti-CTLA-4 antibody is selected from the group consisting of ipilimumab and tremelimumab. The anti-CTLA-4 antibody may also be selected from the compounds disclosed in U.S. Pat. Nos. 6,984,720, 7,605,238, 8,017,114, 8,318,916, 8,784,815, 6,682,736, 7,109,003, 7,132,281, 7,411,057, 8,143,379, 8,491,895 and 8,883,984, all of which are incorporated by reference herein in their entireties for all purposes.

The amino acid sequences of anti-CTLA-4 antibodies referenced in the foregoing are summarized in Table 1.

TABLE 1
Anti-CTLA-4 antibody sequences.

| Sequence | | |
|---|---|---|
| Identifier and | | |
| Description | Sequence (One-Letter Amino Acid Symbols) | |
| SEQ ID NO: 1 | QVQLVESGGG VVQPGRSLRL SCAASGFTFS SYTMHWVRQA PGKGLEWVTF ISYDGNNKYY | 60 |
| ipilimumab | ADSVKGRFTI SRDNSKNTLY LQMNSLRAED TAIYYCARTG WLGPFDYWGQ GTLVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKRVEPKS CDKTHTCPPC PAPELLGGPS | 240 |
| | VFLFPPKPKD TLMISRTPEV TCVVVDVSHE DPEVKFNWYV DGVEVHNAKT KPREEQYNST | 300 |
| | YRVVSVLTVL HQDWLNGKEY KCKVSNKALP APIEKTISKA KGQPREPQVY TLPPSRDELT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPGK | 448 |
| SEQ ID NO: 2 | EIVLTQSPGT LSLSPGERAT LSCRASQSVG SSYLAWYQQK PGQAPRLLIY GAFSRATGIP | 60 |
| ipilimumab | DRFSGSGSGT DFTLTISRLE PEDFAVYYCQ QYGSSPWTFG QGTKVEIKRT VAAPSVFIFP | 120 |
| light chain | PSDEQLKSGT ASVVCLLNNF YPREAKVQWK VDNALQSGNS QESVTEQDSK DSTYSLSSTL | 180 |
| | TLSKADYEKH KVYACEVTHQ GLSSPVTKSF NRGEC | 215 |
| SEQ ID NO: 3 | QVQLVESGGG VVQPGRSLRL SCAASGFTFS SYTMHWVRQA PGKGLEWVTF ISYDGNNKYY | 60 |
| ipilimumab | ADSVKGRFTI SRDNSKNTLY LQMNSLRAED TAIYYCARTG WLGPFDYWGQ GTLVTVSS | 118 |
| variable heavy | | |
| chain | | |
| SEQ ID NO: 4 | EIVLTQSPGT LSLSPGERAT LSCRASQSVG SSYLAWYQQK PGQAPRLLIY GAFSRATGIP | 60 |
| ipilimumab | DRFSGSGSGT DFTLTISRLE PEDFAVYYCQ QYGSSPWTFG QGTKVEIK | 108 |
| variable light | | |
| chain | | |
| SEQ ID NO: 5 | SYTMH | 5 |
| ipilimumab | | |
| variable heavy | | |
| chain CDR1 | | |
| SEQ ID NO: 6 | FISYDGNNKY YADSVKG | 17 |
| ipilimumab | | |
| variable heavy | | |
| chain CDR2 | | |
| SEQ ID NO: 7 | TGWLGPFDY | 9 |
| ipilimumab | | |
| variable heavy | | |
| chain CDR3 | | |
| SEQ ID NO: 8 | RASQSVGSSYLA | 12 |
| ipilimumab | | |
| variable heavy | | |
| chain CDR1 | | |
| SEQ ID NO: 9 | GAFSRAT | 7 |
| ipilimumab | | |
| variable heavy | | |
| chain CDR2 | | |
| SEQ ID NO: 10 | QQYGSSPWT | 9 |
| ipilimumab | | |
| variable heavy | | |
| chain CDR3 | | |
| SEQ ID NO: 11 | QVQLVESGGG VVQPGRSLRL SCAASGFTFS SYGMHWVRQA PGKGLEWVAV IWYDGSNKYY | 60 |
| tremelimumab | ADSVKGRFTI SRDNSKNTLY LQMNSLRAED TAVYYCARDP RGATLYYYYY GMDVWGQGTT | 120 |
| heavy chain | VTVSSASTKG PSVFPLAPCS RSTSESTAAL GCLVKDYFPE PVTVSWNSGA LTSGVHTFPA | 180 |
| | VLQSSGLYSL SSVVTVPSSN FGTQTYTCNV DHKPSNTKVD KTVERKCCVE CPPCPAPPVA | 240 |
| | GPSVFLFPPK PKDTLMISRT PEVTCVVVDV SHEDPEVQFN WYVDGVEVHN AKTKPREEQF | 300 |
| | NSTFRVVSVL TVVHQDWLNG KEYKCKVSNK GLPAPIEKTI SKTKGQPREP QVYTLPPSRE | 360 |
| | EMTKNQVSLT CLVKGFYPSD IAVEWESNGQ PENNYKTTPP MLDSDGSFFL YSKLTVDKSR | 420 |
| | WQQGNVFSCS VMHEALHNHY TQKSLSLSPG K | 451 |
| SEQ ID NO: 12 | DIQMTQSPSS LSASVGDRVT ITCRASQSIN SYLDWYQQKP GKAPKLLIYA ASSLQSGVPS | 60 |
| tremelimumab | RFSGSGSGTD FTLTISSLQP EDFATYYCQQ YYSTPFTFGP GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 13 | QVQLVESGGG VVQPGRSLRL SCAASGFTFS SYGMHWVRQA PGKGLEWVAV IWYDGSNKYY | 60 |
| tremelimumab | ADSVKGRFTI SRDNSKNTLY LQMNSLRAED TAVYYCARDP RGATLYYYYY GMDVWGQGTT | 120 |
| variable heavy | VTVSS | 125 |
| chain | | |
| SEQ ID NO: 14 | DIQMTQSPSS LSASVGDRVT ITCRASQSIN SYLDWYQQKP GKAPKLLIYA ASSLQSGVPS | 60 |
| tremelimumab | RFSGSGSGTD FTLTISSLQP EDFATYYCQQ YYSTPFTFGP GTKVEIK | 107 |
| variable light | | |
| chain | | |
| SEQ ID NO: 15 | GFTFSSYGMH | 10 |
| tremelimumab | | |
| variable heavy | | |
| chain CDR1 | | |
| SEQ ID NO: 16 | VIWYDGSNKYYADSV | 15 |
| tremelimumab | | |
| variable heavy | | |
| chain CDR2 | | |
| SEQ ID NO: 17 | DPRGATLYYYYYGMDV | 16 |
| tremelimumab | | |
| variable heavy | | |
| chain CDR3 | | |
| SEQ ID NO: 18 | RASQSINSYL D | 11 |
| tremelimumab | | |
| variable light | | |
| chain CDR1 | | |
| SEQ ID NO: 19 | AASSLQS | 7 |
| tremelimumab | | |
| variable light | | |
| chain CDR2 | | |
| SEQ ID NO: 20 | QQYYSTPFT | 9 |
| tremelimumab | | |
| variable light | | |
| chain CDR3¹ | | |

¹Position 3 may alternatively be T or S, position 4 may be F or L, position 5 may be T or S, and position 7 may be H, S, or T.

### PD-1/L1 Inhibitors

In some embodiments, the methods and/or compositions described herein include a combination of an ER antagonist and one or more immunotherapeutic agents. In some embodiments, the immunotherapeutic agents may include one or more programmed death-1 (PD-1) and programmed death ligand 1 (PD-L1) inhibitors.

In some embodiments, the PD-1 or PD-L1 inhibitor (e.g., an ant-PD-1 antibody) for use in combination with ER antagonists is selected from the group consisting of nivolumab, pembrolizumab, pidilizumab, durvalumab, atezolizumab, avelumab, and any fragment, derivative, conjugate, variant, radioisotope-labeled complex, or biosimilar thereof.

In an embodiment, an anti-PD-1 antibody comprises nivolumab (also known as OPDIVO and commercially available from Bristol-Myers Squibb Co.), or biosimilars, antigen-binding fragments, conjugates, or variants thereof. Nivolumab is referred to as 5C4 in International Patent Publication No. WO 2006/121168. Nivolumab is assigned Chemical Abstracts Service (CAS) registry number 946414-94-4 and is also known as BMS-936558, MDX-1106 or ONO-4538. Nivolumab is a fully human IgG4 antibody blocking the PD-1 receptor. The clinical safety and efficacy of nivolumab in various forms of cancer has been described in Wang et al., Cancer Immunol Res. 2014, 2, 846-56; Page et al., Ann. Rev. Med., 2014, 65, 185-202; and Weber et al., J. Clin. Oncology, 2013, 31, 4311-4318. The nivolumab monoclonal antibody includes a heavy chain given by SEQ ID NO:21 and a light chain given by SEQ ID NO:22. Nivolumab has intra-heavy chain disulfide linkages at 22-96,140-196, 254-314, 360-418, 22′-96″, 140″-196″, 254″-314″, and 360″-418″; intra-light chain disulfide linkages at 23′-88′, 134′-194′, 23′″-88″, and 134′″-194′″; inter-heavy-light chain disulfide linkages at 127-214′, 127″-214′″, inter-heavy-heavy chain disulfide linkages at 219-219″ and 222-222″; and N-glycosylation sites (H CH₂ 84.4) at 290, 290″.

In an embodiment, the anti-PD-1 antibody is an anti-PD-1 biosimilar monoclonal antibody approved by drug regulatory authorities with reference to nivolumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is nivolumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is nivolumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab.

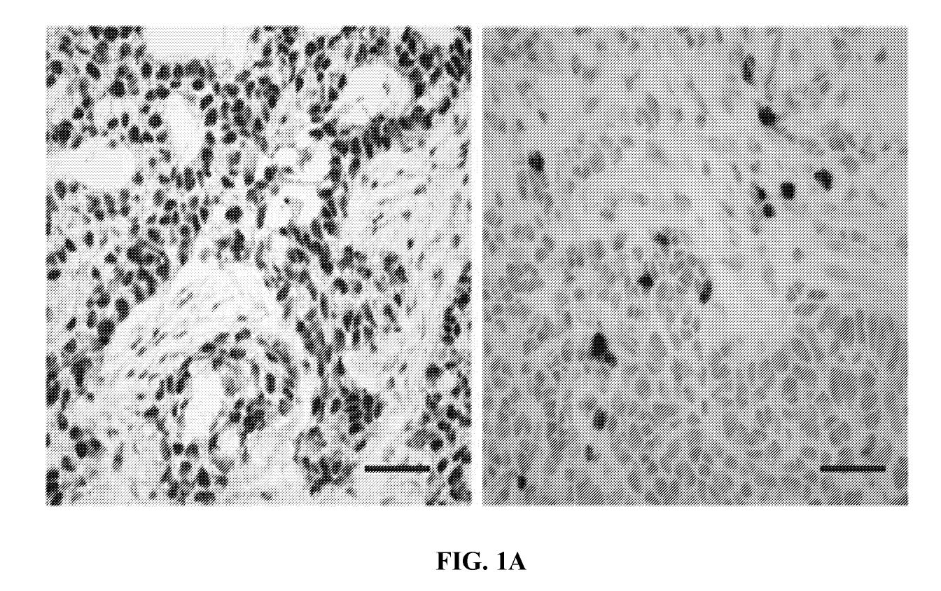

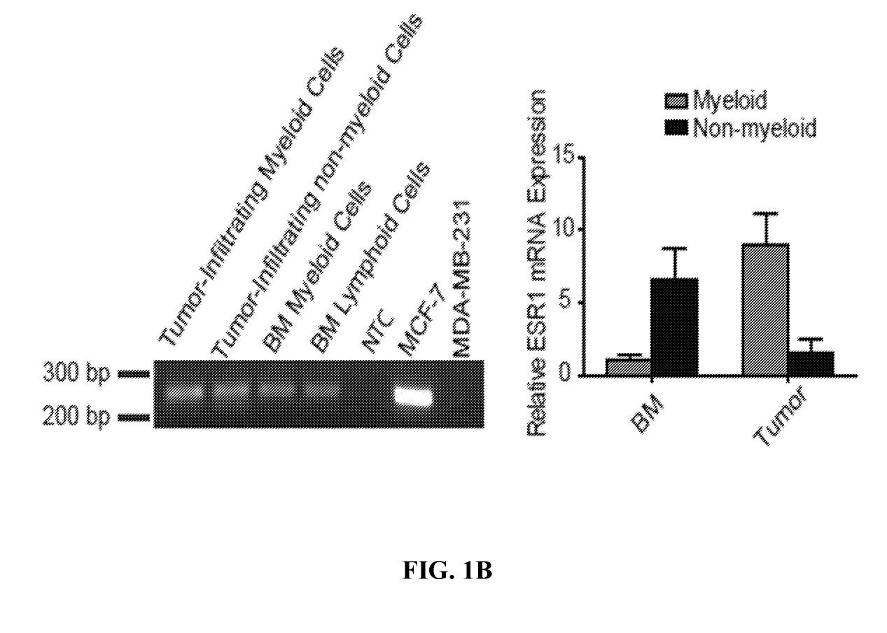

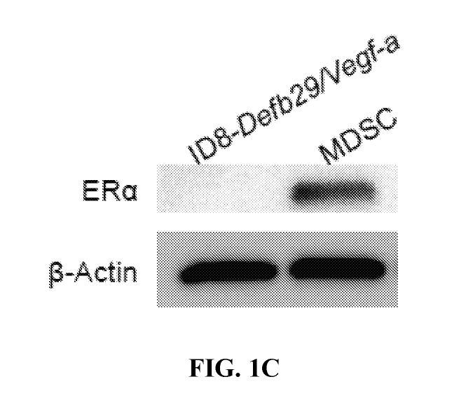

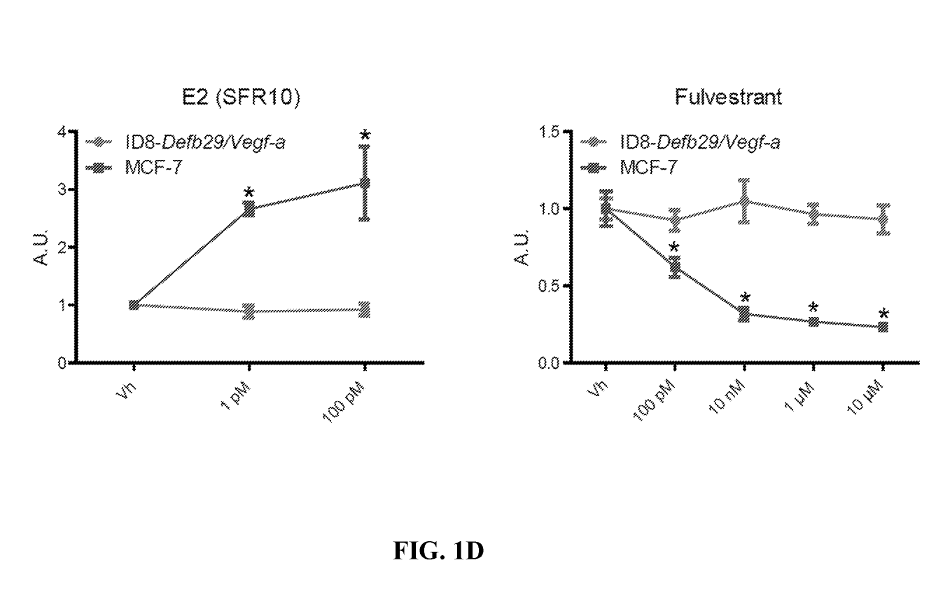

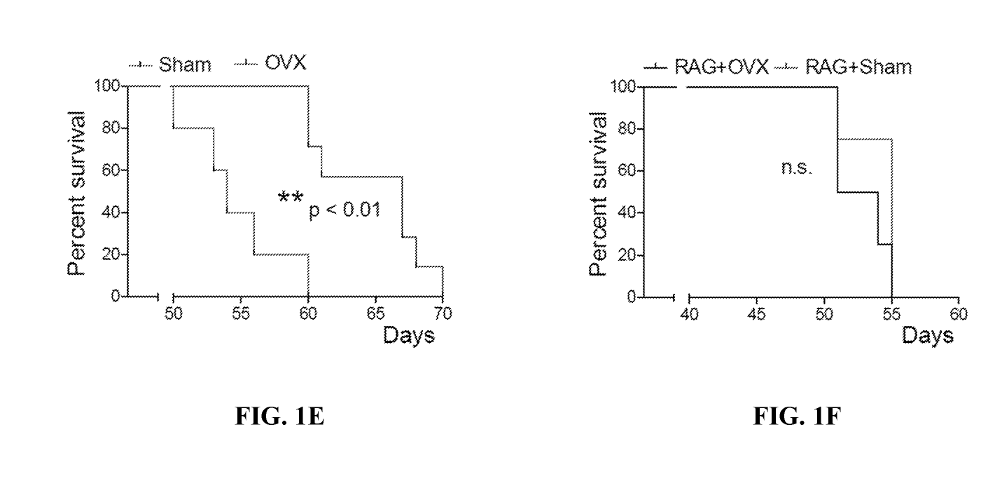

In an embodiment, the anti-PD-1 antibody is an antibody disclosed and/or prepared according to U.S. Pat. No. 8,008,449 or U.S. Patent Application Publication Nos. 2009/0217401 A1 or 2013/0133091 A1, the disclosures of which are specifically incorporated by reference herein. For example, in an embodiment, the monoclonal antibody includes 5C4 (referred to herein as nivolumab), 17D8, 2D3, 4H1, 4A11, 7D3, and 5F4, described in U.S. Pat. No. 8,008,449, the disclosures of which are hereby incorporated by reference. The PD-1 antibodies 17D8, 2D3, 4H1, 5C4, and 4A11, are all directed against human PD-1, bind specifically to PD-1 and do not bind to other members of the CD28 family. The sequences and CDR regions for these antibodies are provided in U.S. Pat. No. 8,008,449, in particular in FIG. 1 through FIG. 12; the disclosures of which are incorporated by reference herein.

In another embodiment, the anti-PD-1 antibody comprises pembrolizumab (also known as KEYTRUDA), which is commercially available from Merck, or antigen-binding fragments, conjugates, or variants thereof. Pembrolizumab is assigned CAS registry number 1374853-91-4 and is also known as lambrolizumab, MK-3475, and SCH-900475. The structure, properties, uses, and preparation of pembrolizumab are described in International Patent Publication No. WO 2008/156712 A1, U.S. Pat. No. 8,354,509 and U.S. Patent Application Publication Nos. US 2010/0266617 A1, US 2013/0108651 A1, and US 2013/0109843 A2, the disclosures of which are incorporated herein by reference. Pembrolizumab has an immunoglobulin G4, anti-(human protein PDCD1 (programmed cell death 1)) (human-Mus musculus monoclonal heavy chain), disulfide with human-Mus musculus monoclonal light chain, dimer structure. The structure of pembrolizumab may also be described as immunoglobulin G4, anti-(human programmed cell death 1); humanized mouse monoclonal [228-L-proline(H10-S>P)]γ4 heavy chain (134-218′)-disulfide with humanized mouse monoclonal κ light chain dimer (226-226″:229-229″)-bisdisulfide. The clinical safety and efficacy of pembrolizumab in various forms of cancer is described in Fuerst, Oncology Times, 2014, 36, 35-36; Robert et al., Lancet, 2014, 384, 1109-17; and Thomas et al., Exp. Opin. Biol. Ther., 2014, 14, 1061-1064. In an embodiment, the pembrolizumab monoclonal antibody includes a heavy chain given by SEQ ID NO:31 and a light chain given by SEQ ID NO:32, and includes the following disulfide bridges: 22-96, 22″-96″, 23′-92′, 23′″-92″, 134-218′, 134″-218′″, 138′-198′, 138′″-198′″, 147-203, 147″-203″, 226-226″, 229-229″, 261-321, 261″-321″, 367-425, and 367″-425″, and the following glycosylation sites (N): Asn-297 and Asn-297″. Pembrolizumab is an IgG4/kappa isotype with a stabilizing S228P mutation in the Fc region; insertion of this mutation in the IgG4 hinge region prevents the formation of half molecules typically observed for IgG4 antibodies. Pembrolizumab is heterogeneously glycosylated at Asn297 within the Fc domain of each heavy chain, yielding a molecular weight of approximately 149 kDa for the intact antibody. The dominant glycoform of pembrolizumab is the fucosylated agalacto diantennary glycan form (G0F).

In an embodiment, the anti-PD-1 antibody is an anti-PD-1 biosimilar monoclonal antibody approved by drug regulatory authorities with reference to pembrolizumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pembrolizumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pembrolizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab.

In an embodiment, the anti-PD-1 antibody is an antibody disclosed in U.S. Pat. No. 8,354,509 or U.S. Patent Application Publication Nos. 2010/0266617 A1, 2013/0108651 A1, 2013/0109843 A2, the disclosures of which are specifically incorporated by reference herein.

In an embodiment, the anti-PD-1 antibody is pidilizumab, which is also known as CT-011 (CureTech Ltd.), and which is disclosed in U.S. Pat. No. 8,686,119 B2, the disclosures of which are specifically incorporated by reference herein. The efficacy of pidilizumab in the treatment of cancers, such as hematological malignancies, is described in Berger, et al., Clin. Cancer Res. 2008, 14, 3044-51. The pidilizumab monoclonal antibody includes a heavy chain given by SEQ ID NO:41 and a light chain given by SEQ ID NO:42. Pidilizumab has intra-heavy chain disulfide linkages at 22-96, 144-200, 261-321, 367-425, 22″-96″, 144″-200″, 261″-321″, and 367″-425″; intra-light chain disulfide linkages at 23′-87′, 133′-193′, 23′-87′, and 133′″-193″; inter-heavy-light chain disulfide linkages at 220-213′ and 220″-213′″, inter-heavy-heavy chain disulfide linkages at 226-226″ 229-229″; and N-glycosylation sites (H CH₂ 84.4) at 297, 297″.

In an embodiment, the anti-PD-1 antibody is an anti-PD-1 biosimilar monoclonal antibody approved by drug regulatory authorities with reference to pidilizumab. In an embodiment, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pidilizumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pidilizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pidilizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pidilizumab.

In an embodiment, anti-PD-1 antibodies and other PD-1 inhibitors include those described in U.S. Pat. Nos. 8,287,856, 8,580,247, and 8,168,757 and U.S. Patent Application Publication Nos. 2009/0028857 A1, 2010/0285013 A1, 2013/0022600 A1, and 2011/0008369 A1, the teachings of which are hereby incorporated by reference. In another embodiment, antibodies that compete with any of these antibodies for binding to PD-1 are also included. In another embodiment, the anti-PD-1 antibody is an antibody disclosed in U.S. Pat. No. 8,735,553 B1, the disclosures of which are incorporated herein by reference.

In an embodiment, the anti-PD-1 antibody is a commercially-available monoclonal antibody, such as anti-m-PD-1 clones J43 (Cat # BE0033-2) and RMP1-14 (Cat # BE0146) (Bio X Cell, Inc.). A number of commercially-available anti-PD-1 antibodies are known to one of ordinary skill in the art.

In an embodiment, the PD-1 inhibitor may be a small molecule or a peptide, or a peptide derivative, such as those described in U.S. Pat. Nos. 8,907,053; 9,096,642; and 9,044,442 and U.S. Patent Application Publication No. 2015/0087581; 1,2,4 oxadiazole compounds and derivatives such as those described in U.S. Patent Application Publication No. 2015/0073024; cyclic peptidomimetic compounds and derivatives such as those described in U.S. Patent Application Publication No. 2015/0073042; cyclic compounds and derivatives such as those described in U.S. Patent Application Publication No. 2015/0125491; 1,3,4 oxadiazole and 1,3,4 thiadiazole compounds and derivatives such as those described in International Patent Application Publication No. WO 2015/033301; peptide-based compounds and derivatives such as those described in International Patent Application Publication Nos. WO 2015/036927 and WO 2015/04490, or a macrocyclic peptide-based compounds and derivatives such as those described in U.S. Patent Application Publication No. 2014/0294898; the disclosures of each of which are hereby incorporated by reference in their entireties.

The anti-PD-1 antibody sequences discussed and referenced in some of the foregoing embodiments are summarized in Table 2.

TABLE 2
Anti-PD-1 antibody amino acid sequences.

| Identifier | Sequence (One-Letter Amino Acid Symbols) | |
|---|---|---|
| SEQ ID NO: 21 | QVQLVESGGG VVQPGRSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSSASTKGPS | 120 |
| heavy chain | VFPLAPCSRS TSESTAALGC LVKDYFPEPV TVSWNSGALT SGVHTFPAVL QSSGLYSLSS | 180 |
| | VVTVPSSSLG TKTYTCNVDH KPSNTKVDKR VESKYGPPCP PCPAPEFLGG PSVFLFPPKP | 240 |
| | KDTLMISRTP EVTCVVVDVS QEDPEVQFNW YVDGVEVHNA KTKPREEQFN STYRVVSVLT | 300 |
| | VLHQDWLNGK EYKCKVSNKG LPSSIEKTIS KAKGQPREPQ VYTLPPSQEE MTKNQVSLTC | 360 |
| | LVKGFYPSDI AVEWESNGQP ENNYKTTPPV LDSDGSFFLY SRLTVDKSRW QEGNVFSCSV | 420 |
| | MHEALHNHYT QKSLSLSLGK | 440 |
| SEQ ID NO: 22 | EIVLTQSPAT LSLSPGERAT LSCRASQSVS SYLAWYQQKP GQAPRLLIYD ASNRATGIPA | 60 |
| nivolumab | RFSGSGSGTD FTLTISSLEP EDFAVYYCQQ SSNWPRTFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 23 | QVQLVESGGG VVQPGRSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSS | 113 |
| variable heavy | | |
| chain | | |
| SEQ ID NO: 24 | EIVLTQSPAT LSLSPGERAT LSCRASQSVS SYLAWYQQKP GQAPRLLIYD ASNRATGIPA | 60 |
| nivolumab | RFSGSGSGTD FTLTISSLEP EDFAVYYCQQ SSNWPRTFGQ GTKVEIK | 107 |
| variable light | | |
| chain | | |
| SEQ ID NO: 28 | NSGMH | 5 |
| nivolumab | | |
| heavy chain | | |
| CDR1 | | |
| SEQ ID NO: 26 | VIWYDGSKRY YADSVKG | 17 |
| nivolumab | | |
| heavy chain | | |
| CDR2 | | |
| SEQ ID NO: 27 | NDDY | 4 |
| nivolumab | | |
| heavy chain | | |
| CDR3 | | |
| SEQ ID NO: 28 | RASQSVSSYL A | 11 |
| nivolumab | | |
| light chain | | |
| CDR1 | | |
| SEQ ID NO: 29 | DASNRAT | 7 |
| nivolumab | | |
| light chain | | |
| CDR2 | | |
| SEQ ID NO: 30 | QQSSNWPRT | 9 |
| nivolumab | | |
| light chain | | |
| CDR3 | | |
| SEQ ID NO: 31 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| heavy chain | ASTKGPSVFP LAPCSRSTSE STAALGCLVK DYFPEPVTVS WNSGALTSGV HTFPAVLQSS | 180 |
| | GLYSLSSVVT VPSSSLGTKT YTCNVDHKPS NTKVDKRVES KYGPPCPPCP APEFLGGPSV | 240 |
| | FLFPPKPKDT LMISRTPEVT CVVVDVSQED PEVQFNWYVD GVEVHNAKTK PREEQFNSTY | 300 |
| | RVVSVLTVLH QDWLNGKEYK CKVSNKGLPS SIEKTISKAK GQPREPQVYT LPPSQEEMTK | 360 |
| | NQVSLTCLVK GFYPSDIAVE WESNGQPENN YKTTPPVLDS DGSFFLYSRL TVDKSRWQEG | 420 |
| | NVFSCSVMHE ALHNHYTQKS LSLSLGK | 447 |
| SEQ ID NO: 32 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI KRTVAAPSVF | 120 |
| light chain | IFPPSDEQLK SGTASVVCLL NNFYPREAKV QWKVDNALQS GNSQESVTEQ DSKDSTYSLS | 180 |
| | STLTLSKADY EKHKVYACEV THQGLSSPVT KSFNRGEC | 218 |
| SEQ ID NO: 33 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| variable heavy | | |
| chain | | |
| SEQ ID NO: 34 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI K | 111 |
| variable light | | |
| chain | | |
| SEQ ID NO: 38 | NYYMY | 5 |
| pembrolizumab | | |
| heavy chain | | |
| CDR1 | | |
| SEQ ID NO: 36 | GINPSNGGTN FNEKFK | 16 |
| pembrolizumab | | |
| heavy chain | | |
| CDR2 | | |
| SEQ ID NO: 37 | RDYRFDMGFD Y | 11 |
| pembrolizumab | | |
| heavy chain | | |
| CDR3 | | |
| SEQ ID NO: 38 | RASKGVSTSG YSYLH | 15 |
| pembrolizumab | | |
| light chain | | |
| CDR1 | | |
| SEQ ID NO: 39 | LASYLES | 7 |
| pembrolizumab | | |
| light chain | | |
| CDR2 | | |
| SEQ ID NO: 40 | QHSRDLPLT | 9 |
| pembrolizumab | | |
| light chain | | |
| CDR3 | | |
| SEQ ID NO: 41 | QVQLVQSGSE LKKPGASVKI SCKASGYTFT NYGMNWVRQA PGQGLQWMGW INTDSGESTY | 60 |
| pidilizumab | AEEFKGRFVF SLDTSVNTAY LQITSLTAED TGMYFCVRVG YDALDYWGQG TLVTVSSAST | 120 |
| heavy chain | KGPSVFPLAP SSKSTSGGTA ALGCLVKDYF PEPVTVSWNS GALTSGVHTF PAVLQSSGLY | 180 |
| | SLSSVVTVPS SSLGTQTYIC NVNHKPSNTK VDKRVEPKSC DKTHTCPPCP APELLGGPSV | 240 |
| | FLFPPKPKDT LMISRTPEVT CVVVDVSHED PEVKFNWYVD GVEVHNAKTK PREEQYNSTY | 300 |
| | RVVSVLTVLH QDWLNGKEYK CKVSNKALPA PIEKTISKAK GQPREPQVYT LPPSREEMTK | 360 |
| | NQVSLTCLVK GFYPSDIAVE WESNGQPENN YKTTPPVLDS DGSFFLYSKL TVDKSRWQQG | 420 |
| | NVFSCSVMHE ALHNHYTQKS LSLSPGK | 447 |
| SEQ ID NO: 42 | EIVLTQSPSS LSASVGDRVT ITCSARSSVS YMHWFQQKPG KAPKLWIYRT SNLASGVPSR | 60 |
| pidilizumab | FSGSGSGTSY CLTINSLQPE DFATYYCQQR SSFPLTFGGG TKLEIKRTVA APSVFIFPPS | 120 |
| light chain | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 43 | QVQLVQSGSE LKKPGASVKI SCKASGYTFT NYGMNWVRQA PGQGLQWMGW INTDSGESTY | 60 |
| pidilizumab | AEEFKGRFVF SLDTSVNTAY LQITSLTAED TGMYFCVRVG YDALDYWGQG TLVTVSS | 117 |
| variable heavy | | |
| chain | | |
| SEQ ID NO: 44 | EIVLTQSPSS LSASVGDRVT ITCSARSSVS YMHWFQQKPG KAPKLWIYRT SNLASGVPSR | 60 |
| pidilizumab | FSGSGSGTSY CLTINSLQPE DFATYYCQQR SSFPLTFGGG TKLEIK | 106 |
| variable light | | |
| chain | | |

The PD-L1 inhibitor may be any PD-L1 inhibitor or blocker known in the art. In particular, it is one of the PD-L1 inhibitors or blockers described in more detail in the following paragraphs. The terms “inhibitor” and “blocker” are used interchangeably herein in reference to PD-L1 inhibitors. For avoidance of doubt, references herein to a PD-L1 inhibitor that is an antibody may refer to a compound or fragment, derivative, conjugate, variant, radioisotope-labeled complex, or biosimilar thereof. For avoidance of doubt, references herein to a PD-L1 inhibitor may refer to a compound or a pharmaceutically acceptable salt, ester, solvate, hydrate, cocrystal, or prodrug thereof.

In an embodiment, the anti-PD-L1 antibody is durvalumab, also known as MEDI4736 (which is commercially available from Medimmune, LLC), or antigen-binding fragments, conjugates, or variants thereof. In an embodiment, the anti-PD-L1 antibody is an antibody disclosed in U.S. Pat. No. 8,779,108 or U.S. Patent Application Publication No. 2013/0034559, the disclosures of which are specifically incorporated by reference herein. The clinical efficacy of durvalumab (MEDI4736, SEQ ID NO:45 and SEQ ID NO:46) has been described in: Page et al., Ann. Rev. Med., 2014, 65, 185-202; Brahmer et al., J. Clin. Oncol. 2014, 32, 5s (supplement, abstract 8021); and McDermott et al., Cancer Treatment Rev., 2014, 40, 1056-64. The durvalumab monoclonal antibody includes disulfide linkages at 22-96, 22″-96″, 23′-89′, 23′″-89′″, 135′-195′, 135′″-195′″, 148-204, 148″-204″, 215′-224, 215′″-224″, 230-230″, 233-233″, 265-325, 265″-325″, 371-429, and 371″-429′; and N-glycosylation sites at Asn-301 and Asn-301″.

In an embodiment, the anti-PD-L1 antibody is an anti-PD-L1 biosimilar monoclonal antibody approved by drug regulatory authorities with reference to durvalumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is durvalumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is durvalumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab.

In an embodiment, anti-PD-L1 antibodies and other PD-L1 inhibitors include those described in U.S. Pat. No. 8,779,108 and U.S. Patent Application Publication No. 2013/0034559A1, the disclosures of which are hereby incorporated by reference. In another embodiment, antibodies that compete with any of these antibodies for binding to PD-L1 are also included.

In an embodiment, the anti-PD-L1 antibody is atezolizumab, also known as MPDL3280A or RG7446 (commercially available from Genentech, Inc.), or antigen-binding fragments, conjugates, or variants thereof. In an embodiment, the anti-PD-L1 antibody is an antibody disclosed in U.S. Pat. No. 8,217,149, the disclosure of which is specifically incorporated by reference herein. In an embodiment, the anti-PD-L1 antibody is an antibody disclosed in U.S. Patent Application Publication Nos. 2010/0203056 A1, 2013/0045200 A1, 2013/0045201 A1, 2013/0045202 A1, or 2014/0065135 A1, the disclosures of which are specifically incorporated by reference herein. The atezolizumab monoclonal antibody includes a heavy chain given by SEQ ID NO:55 and a light chain given by SEQ ID NO:56. Atezolizumab has intra-heavy chain disulfide linkages (C23-C104) at 22-96, 145-201, 262-322, 368-426, 22″-96″, 145″-201″, 262″-322″, and 368″-426″; intra-light chain disulfide linkages (C23-C104) at 23′-88′, 134′-194′, 23′″-88″, and 134′″-194′″; intra-heavy-light chain disulfide linkages (h 5-CL 126) at 221-214′ and 221″-214′″; intra-heavy-heavy chain disulfide linkages (h 11, h 14) at 227-227″ and 230-230″; and N-glycosylation sites (H CH₂ N84.4>A) at 298 and 298′.

In an embodiment, the anti-PD-L1 antibody is an anti-PD-L1 biosimilar monoclonal antibody approved by drug regulatory authorities with reference to atezolizumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is atezolizumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is atezolizumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab.

In an embodiment, the anti-PD-L1 antibody is avelumab, also known as MSB0010718C (commercially available from Merck KGaA/EMD Serono), or antigen-binding fragments, conjugates, or variants thereof. In an embodiment, the anti-PD-L1 antibody is an antibody disclosed in U.S. Patent Application Publication No. US 2014/0341917 A1, the disclosure of which is specifically incorporated by reference herein. The avelumab monoclonal antibody includes a heavy chain given by SEQ ID NO:65 and a light chain given by SEQ ID NO:66. Avelumab has intra-heavy chain disulfide linkages (C23-C104) at 22-96, 147-203, 264-324, 370-428, 22″-96″, 147″-203″, 264″-324″, and 370″-428″; intra-light chain disulfide linkages (C23-C104) at 22′-90′, 138′-197′, 22′″-90′″, and 138′″-197′″; intra-heavy-light chain disulfide linkages (h 5-CL 126) at 223-215′ and 223″-215′″; intra-heavy-heavy chain disulfide linkages (h 11, h 14) at 229-229″ and 232-232″; N-glycosylation sites (H CH₂ N84.4) at 300, 300″; fucosylated complex bi-antennary CHO-type glycans; and H CHS K2 C-terminal lysine clipping at 450 and 450′.

In an embodiment, the anti-PD-L1 antibody is an anti-PD-L1 biosimilar monoclonal antibody approved by drug regulatory authorities with reference to avelumab. In an embodiment, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is avelumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is avelumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab.

In an embodiment, anti-PD-L1 antibodies and other PD-L1 inhibitors include those described in U.S. Patent Application Publication No. 2014/0341917 A1, the disclosure of which is hereby incorporated by reference. In another embodiment, antibodies that compete with any of these antibodies for binding to PD-L1 are also included.

In an embodiment, the anti-PD-L1 antibody is MDX-1105, also known as BMS-935559, which is disclosed in U.S. Pat. No. 7,943,743 B2, the disclosures of which are specifically incorporated by reference herein. In an embodiment, the anti-PD-L1 antibody is selected from the anti-PD-L1 antibodies disclosed in U.S. Pat. No. 7,943,743 B2, which are specifically incorporated by reference herein.

In an embodiment, the anti-PD-L1 antibody is a commercially-available monoclonal antibody, such as INVIVOMAB anti-m-PD-L1 clone 10F.9G2 (Catalog # BE0101, Bio X Cell, Inc.). In an embodiment, the anti-PD-L1 antibody is a commercially-available monoclonal antibody, such as AFFYMETRIX EBIOSCIENCE (MIH1). A number of commercially-available anti-PD-L1 antibodies are known to one of ordinary skill in the art.

The anti-PD-L1 antibody sequences referenced in some of the foregoing embodiments are summarized in Table 3.

TABLE 3
Anti-PD-L1 antibody amino acid sequences.

| Identifier | Sequence (One-Letter Amino Acid Symbols) | |
|---|---|---|
| SEQ ID NO: 45 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| (MEDI4736) | SASTKGPSVF PLAPSSKSTS GGTAALGCLV KDYFPEPVTV SWNSGALTSG VHTFPAVLQS | 180 |
| heavy chain | SGLYSLSSVV TVPSSSLGTQ TYICNVNHKP SNTKVDKRVE PKSCDKTHTC PPCPAPEFEG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPASIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 46 | EVQLVESGGG LVQPGGSLRL SCAASGFTFS RYWMSWVRQA PGKGLEWVAN EIVLTQSPGT | 60 |
| durvalumab | LSLSPGERAT LSCRASQRVS SSYLAWYQQK PGQAPRLLIY DASSRATGIP DRFSGSGSGT | 120 |
| (MEDI4736) | DFTLTISRLE PEDFAVYYCQ QYGSLPWTFG QGTKVEIKRT VAAPSVFIFP PSDEQLKSGT | 180 |
| light chain | ASVVCLLNNF YPREAKVQWK VDNALQSGNS QESVTEQDSK DSTYSLSSTL TLSKADYEKH | 240 |
| | KVYACEVTHQ GLSSPVTKSF NRGEC | 265 |
| SEQ ID NO: 47 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| variable | S | 121 |
| heavy chain | | |
| SEQ ID NO: 48 | EIVLTQSPGT LSLSPGERAT LSCRASQRVS SSYLAWYQQK PGQAPRLLIY DASSRATGIP | 60 |
| durvalumab | DRFSGSGSGT DFTLTISRLE PEDFAVYYCQ QYGSLPWTFG QGTKVEIK | 108 |
| variable | | |
| light chain | | |
| SEQ ID NO: 49 | RYWMS | 5 |
| durvalumab | | |
| heavy chain | | |
| CDR1 | | |
| SEQ ID NO: 50 | NIKQDGSEKY YVDSVKG | 17 |
| durvalumab | | |
| heavy chain | | |
| CDR2 | | |
| SEQ ID NO: 51 | EGGWFGELAF DY | 12 |
| durvalumab | | |
| heavy chain | | |
| CDR3 | | |
| SEQ ID NO: 52 | RASQRVSSSY LA | 12 |
| durvalumab | | |
| light chain | | |
| CDR1 | | |
| SEQ ID NO: 53 | DASSRAT | 7 |
| durvalumab | | |
| light chain | | |
| CDR2 | | |
| SEQ ID NO: 54 | QQYGSLPWT | 9 |
| durvalumab | | |
| light chain | | |
| CDR3 | | |
| SEQ ID NO: 55 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSSAS | 120 |
| (MPDL3280A) | TKGPSVFPLA PSSKSTSGGT AALGCLVKDY FPEPVTVSWN SGALTSGVHT FPAVLQSSGL | 180 |
| heavy chain | YSLSSVVTVP SSSLGTQTYI CNVNHKPSNT KVDKKVEPKS CDKTHTCPPC PAPELLGGPS | 240 |
| | VFLFPPKPKD TLMISRTPEV TCVVVDVSHE DPEVKFNWYV DGVEVHNAKT KPREEQYAST | 300 |
| | YRVVSVLTVL HQDWLNGKEY KCKVSNKALP APIEKTISKA KGQPREPQVY TLPPSREEMT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPGK | 448 |
| SEQ ID NO: 56 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKRTV AAPSVFIFPP | 120 |
| (MPDL3280A) | SDEQLKSGTA SVVCLLNNFY PREAKVQWKV DNALQSGNSQ ESVTEQDSKD STYSLSSTLT | 180 |
| light chain | LSKADYEKHK VYACEVTHQG LSSPVTKSFN RGEC | 214 |
| SEQ ID NO: 57 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSA | 118 |
| variable | | |
| heavy chain | | |
| SEQ ID NO: 58 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKR | 108 |
| variable | | |
| light chain | | |
| SEQ ID NO: 59 | GFTFSXSWIH | 10 |
| atezolizumab | | |
| heavy chain | | |
| CDR1 | | |
| SEQ ID NO: 60 | AWIXPYGGSX YYADSVKG | 18 |
| atezolizumab | | |
| heavy chain | | |
| CDR2 | | |
| SEQ ID NO: 61 | RHWPGGFDY | 9 |
| atezolizumab | | |
| heavy chain | | |
| CDR3 | | |
| SEQ ID NO: 62 | RASQXXXTXX A | 11 |
| atezolizumab | | |
| light chain | | |
| CDR1 | | |
| SEQ ID NO: 63 | SASXLXS | 7 |
| atezolizumab | | |
| light chain | | |
| CDR2 | | |
| SEQ ID NO: 64 | QQXXXXPXT | 9 |
| atezolizumab | | |
| light chain | | |
| CDR3 | | |
| SEQ ID NO: 68 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| (MSB0010718C) | ASTKGPSVFP LAPSSKSTSG GTAALGCLVK DYFPEPVTVS WNSGALTSGV HTFPAVLQSS | 180 |
| heavy chain | GLYSLSSVVT VPSSSLGTQT YICNVNHKPS NTKVDKKVEP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 480 |
| SEQ ID NO: 66 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL GQPKANPTVT | 120 |
| (MSB0010718C) | LFPPSSEELQ ANKATLVCLI SDFYPGAVTV AWKADGSPVK AGVETTKPSK QSNNKYAASS | 180 |
| light chain | YLSLTPEQWK SHRSYSCQVT HEGSTVEKTV APTECS | 216 |
| SEQ ID NO: 67 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| variable | | |
| heavy chain | | |
| SEQ ID NO: 68 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL | 110 |
| variable | | |
| light chain | | |
| SEQ ID NO: 69 | SYIMM | 5 |
| avelumab | | |
| heavy chain | | |
| CDR1 | | |
| SEQ ID NO: 70 | SIYPSGGITF YADTVKG | 17 |
| avelumab | | |
| heavy chain | | |
| CDR2 | | |
| SEQ ID NO: 71 | IKLGTVTTVD Y | 11 |
| avelumab | | |
| heavy chain | | |
| CDR3 | | |
| SEQ ID NO: 72 | TGTSSDVGGY NYVS | 14 |
| avelumab | | |
| light chain | | |
| CDR1 | | |
| SEQ ID NO: 73 | DVSNRPS | 7 |
| avelumab | | |
| light chain | | |
| CDR2 | | |
| SEQ ID NO: 74 | SSYTSSSTRV | 10 |
| avelumab | | |
| light chain | | |
| CDR3 | | |

The preparation, properties, and uses of suitable PD-1 and PD-L1 inhibitors are described in, e.g., U.S. Pat. No. 8,008,449 or U.S. Patent Application Publication Nos. 2009/0217401 A1 or 2013/0133091 A1; U.S. Pat. No. 8,354,509 and U.S. Patent Application Publication Nos. 2010/0266617 A1, 2013/0108651 A1, and 2013/0109843 A2; U.S. Pat. Nos. 8,287,856, 8,580,247, and 8,168,757 and U.S. Patent Application Publication Nos. US 2009/0028857 A1, US 2010/0285013 A1, US 2013/0022600 A1, and US 2011/0008369 A1; U.S. Pat. No. 8,779,108 or U.S. Patent Application Publication No. 2013/0034559 A1; U.S. Pat. No. 8,217,149 and U.S. Patent Application Publication Nos. 2010/0203056 A1, 2013/0045200 A1, 2013/0045201 A1, 2013/0045202 A1, or 2014/0065135 A1; and U.S. Patent Application Publication No. 2014/0341917 A1, the disclosures of each of which are incorporated by reference herein.

In an embodiment, a PD-1 or PD-L1 inhibitor selected from the group consisting of nivolimumab, pembrolizumab, pidilizumab, durvalumab, atezolizumab, avelumab, and/or Fab fragments, antigen-binding fragments, derivatives, conjugates, variants, and radioisotope-labeled complexes thereof, is administered to a subject by infusing a dose selected from the group consisting of about 10 mg, about 20 mg, about 25 mg, about 50 mg, about 75 mg, 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, and about 2000 mg. In an embodiment, the PD-1 or PD-L1 inhibitor is administered weekly. In an embodiment, the PD-1 or PD-L1 inhibitor is administered every two weeks. In an embodiment, the PD-1 or PD-L1 inhibitor is administered every three weeks. In an embodiment, the PD-1 or PD-L1 inhibitor is administered monthly. In an embodiment, the PD-1 or PD-L1 inhibitor is administered at a lower initial dose, which is escalated when administered at subsequent intervals administered monthly.

### IDO Inhibitors

In some embodiments, the methods and/or compositions described herein include a combination of an ER antagonist and one or more immunotherapeutic agents. In some embodiments, the immunotherapeutic agents may include one or more indoleamine-2,3-dioxygenase (IDO) inhibitors.

The IDO inhibitor may be any IDO inhibitor known in the art. In particular, it is one of the IDO inhibitors described in more detail in the following paragraphs. In an embodiment, the IDO inhibitor is an indoleamine-2,3-dioxygenase 1 (IDO1) inhibitor. In an embodiment, it is a selective IDO1 inhibitor. In an embodiment, it is an indoleamine-2,3-dioxygenase 2 (ID02) inhibitor. In an embodiment, it is a selective IDO2 inhibitor. In some embodiments, the compositions described herein provide a combination of an IDO inhibitor with an ER antagonist, or methods of using a combination of an IDO inhibitor with an ER antagonist. In some embodiments, the IDO inhibitor is selected from the group consisting of N-(3-bromo-4-fluorophenyl)-N-hydroxy-4-((2-(sulfamoylamino)ethyl)amino)-1,2,5-oxadiazole-3-carboximidamide and 1-methyl-D-tryptophan

In an embodiment, the IDO inhibitor is a compound of Formula (Xa) or (Xb):

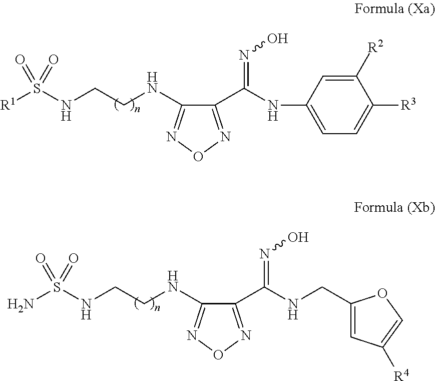

wherein R¹ is NH₂ or CH₃; R² is Cl, Br, CF₃, CH₃, or CN; R³ is H or F; R⁴ is F, Cl, Br, or I; and n is 1 or 2; or a pharmaceutically acceptable salt, solvate, hydrate, cocrystal, or prodrug thereof. The wavy bond [symbol] in Formulas (Xa) and (Xb) represents a mixture of (E)- and (Z)-isomers. The compounds of Formula (Xa) or (Xb) may be synthesized as described in U.S. Pat. No. 8,088,803; the disclosure of which is incorporated herein by reference in its entirety.

In an embodiment, the IDO inhibitor is a compound of Formula (Xa) or Formula (Xb) or a pharmaceutically acceptable salt, solvate, hydrate, cocrystal, or prodrug thereof.

In an embodiment, the IDO inhibitor is a compound selected from those disclosed in U.S. Pat. No. 8,088,803, the disclosure of which is incorporated herein by reference in its entirety, or a pharmaceutically acceptable salt, solvate, hydrate, cocrystal, or prodrug thereof.

In an embodiment, the IDO inhibitor is a compound of Formula (XIa) or (XIb):

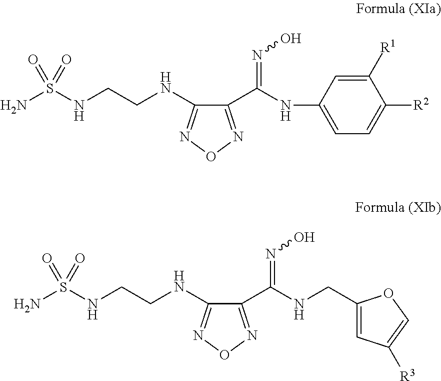

wherein R¹ is Cl, Br, CF₃, or CN; R² is H or F; and R³ is Cl or Br; or a pharmaceutically acceptable salt, solvate, hydrate, cocrystal, or prodrug thereof. The wavy bond [symbol] in Formulas (XIa) and (XIb) represents a mixture of (E)- and (Z)-isomers. The compounds of Formula (XIa) or (XIb) may be synthesized as described in U.S. Pat. No. 8,088,803.

In a preferred embodiment, the IDO inhibitor is a compound of Formula (XIa) or Formula (XIb) or a pharmaceutically acceptable salt, solvate, hydrate, cocrystal, or prodrug thereof.

In a preferred embodiment, the IDO inhibitor is N-(3-bromo-4-fluorophenyl)-N-hydroxy-4-((2-(sulfamoylamino)ethyl)amino)-1,2,5-oxadiazole-3-carboximidamide, or a pharmaceutically acceptable salt, solvate, hydrate, cocrystal, or prodrug thereof. In a preferred embodiment, the IDO inhibitor is a compound of Formula (XII):

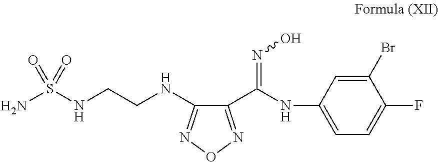 [SMILES: NS(=O)(=O)NCCNc1nonc1C(=NO)Nc1ccc(F)c(Br)c1]

or a pharmaceutically acceptable salt, solvate, hydrate, cocrystal, or prodrug thereof. The wavy bond [symbol] in Formula (XII) represents a mixture of cis- and trans-isomers.

In a preferred embodiment, the IDO inhibitor is (Z)—N-(3-bromo-4-fluorophenyl)-N-hydroxy-4-[2-(sulfamoylamino)ethylamino]-1,2,5-oxadiazole-3-carboxamidine, also known as epacadostat and INCB024360 (Incyte Corp., Wilmington, Del., USA), or a pharmaceutically acceptable salt, solvate, hydrate, cocrystal, or prodrug thereof. In a preferred embodiment, the IDO inhibitor is a compound of Formula (XIII):

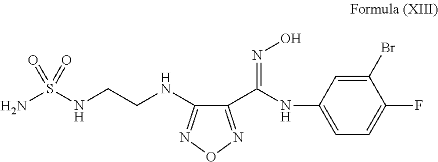 [SMILES: NS(=O)(=O)NCCNc1nonc1/C(=N/O)Nc1ccc(F)c(Br)c1]

or a pharmaceutically acceptable salt, solvate, hydrate, cocrystal, or prodrug thereof. In a preferred embodiment, the IDO inhibitor is 1,2,5-oxadiazole-3-carboximidamide, 4-[[2-[(aminosulfonyl)amino]ethyl]amino]-N-(3-bromo-4-fluorophenyl)-N′-hydroxy-, [C(Z)]—, or a pharmaceutically acceptable salt, solvate, hydrate, cocrystal, or prodrug thereof.

In an embodiment, the IDO inhibitor is a compound selected from the IDO inhibitors disclosed in WO 2015/119944 A1, the disclosures of which are incorporated herein by reference in their entirety; or a pharmaceutically acceptable salt, solvate, hydrate, cocrystal, or prodrug thereof.

In a preferred embodiment, the IDO inhibitor is 1-methyl-tryptophan, or a pharmaceutically acceptable salt, solvate, hydrate, cocrystal, or prodrug thereof. In an embodiment, the IDO inhibitor is a compound of Formula (XIV):

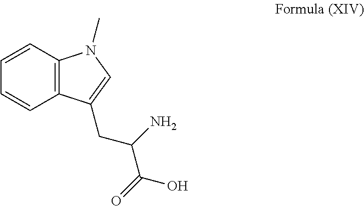 [SMILES: Cn1cc(CC(N)C(=O)O)c2ccccc21]

or a pharmaceutically acceptable salt, solvate, hydrate, cocrystal, or prodrug thereof.

In a preferred embodiment, the IDO inhibitor is 1-methyl-D-tryptophan, also known as indoximod, or a pharmaceutically acceptable salt, solvate, hydrate, cocrystal, or prodrug thereof. In an embodiment, the IDO inhibitor is a compound of Formula (XV):

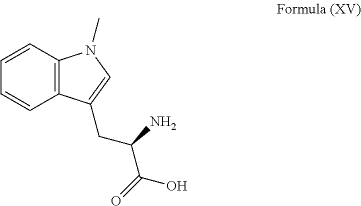 [SMILES: Cn1cc(C[C@@H](N)C(=O)O)c2ccccc21]

or a pharmaceutically acceptable salt, solvate, hydrate, cocrystal, or prodrug thereof. Indoximod has shown activity in human clinical trials of patients with metastatic solid tumors. Soliman, et al., Oncotarget 2014, 5(18), 8136-46.

In an embodiment, the IDO inhibitor is a compound of Formula (XVI):

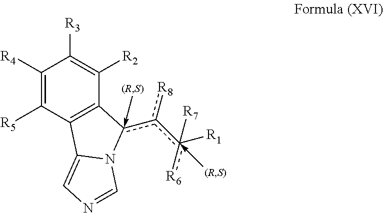

wherein R₁ is selected from saturated or unsaturated cycloalkyl, saturated or unsaturated heterocycloalkyl, aryl, and heteroaryl, each of which is optionally substituted; R₂, R₃, R₄, and R₅ are independently selected from H, —OH, and halogen; R₆ is C═O, ═O, ═N—OH, —NH₂, or —OH; and R₇ and R_{g} are H; or a pharmaceutically acceptable salt, solvate, hydrate, cocrystal, or prodrug thereof. The compounds of Formula (XVI) may be synthesized as described in WO 2012/142237 A1 or US 2014/0066625 A1, the disclosures of which are incorporated herein by reference in their entirety.

In an embodiment, the IDO inhibitor is a compound or pharmaceutically acceptable salt, solvate, hydrate, cocrystal, or prodrug thereof selected from the compounds disclosed in WO 2012/142237 A1 or US 2014/0066625 A1, the disclosures of which are incorporated by reference herein. In an embodiment, the IDO inhibitor is NLG919 (also referred to as GDC-0919 or RG6078) or a pharmaceutically acceptable salt, solvate, hydrate, cocrystal, or prodrug thereof.

### Methods of Treating Cancers and Other Diseases

In some embodiments the invention includes methods of treating cancers and/or solid tumors in a subject with one or more ER antagonists.

In some embodiments, the invention includes methods of treating an ER (−) cancer and/or solid tumor in a subject with ER (+) MDSCs, where the methods further include administering a therapeutically effective amount of an ER antagonist to the subject in need thereof. In some embodiments, the invention includes methods of treating an ER (−) cancer and/or solid tumor in a subject with an elevated population of ER (+) MDSCs, where the methods further include administering a therapeutically effective amount of an ER antagonist to the subject in need thereof.

In some embodiments, the invention includes methods of treating an ER (−) cancer and/or solid tumor in a subject that may include the steps of: (a) obtaining a tissue or bodily fluid (e.g., blood) sample from the subject; (b) analyzing the tissue or bodily fluid sample for MDSCs and testing the MDSCs with an estrogen receptor-specific protein assay, as described herein; (c) determining whether the MDSCs that were tested are ER (+); and (d) administering a therapeutically effective amount of an ER antagonist to the subject in need thereof.

In some embodiments of the methods of the invention, the ER(−) cancer and/or solid tumor may include lung cancer (e.g., non-small cell lung or bronchoalveoloar carcinoma) cells, breast cancer (e.g., mammary carcinoma) cells, endometrial cancer cells, skin cancer (e.g., melanoma) cells, colorectal (e.g., colon and/or rectal) cancer cells, gastric cancer (e.g., gastrointestinal tumor) cells, colorectal cancer cells, brain cancer (e.g., glioblastoma) cells, renal cancer cells, bladder/ureter cancer (e.g., urothelial carcinoma) cells, pancreatic cancer (e.g., pancreatic adenocarcinoma) cells, prostate cancer cells, thyroid cancer (e.g., anaplastic thyroid carcinoma) cells, head and neck cancer (e.g., tongue squamous cell carcinoma or head and neck squamous cell carcinoma) cells, liver cancer cells, lymphoid/splenic cancer cells, or ovarian cancer cells.

In some embodiments, the methods of the invention may include the treatment of a cancer described by Kumar, et al. Trends in Immunology (2016) 37: 208-220, the entirety of which is incorporated herein by reference.

In some embodiments, the methods of the invention may include the treatment of a cancer that may be selected from the group consisting of lung cancer, breast cancer, endometrial cancer, skin cancer, ovarian cancer, gastric cancer, colorectal cancer, brain cancer, renal cancer, bladder cancer, ureter cancer, pancreatic cancer, prostate cancer, thyroid cancer, head and neck cancer, liver cancer, lymphoid cancer, and splenic cancer.

In some embodiments, any of the methods of the invention may further include the administration of a therapeutically effective amount of an immunotherapeutic agent, as described herein.

In some embodiments, the methods of the invention may include testing a subject to determine if they have an elevated level of estradiol and/or estrogen. In some embodiments, the subject may have an elevated level of estradiol and/or estrogen.

Efficacy of the compounds and combinations of compounds described herein in treating, preventing and/or managing the indicated diseases or disorders can be tested using various models known in the art, which provide guidance for treatment of human disease. For example, models for determining efficacy of treatments for ovarian cancer are described, e.g., in Mullany et al., Endocrinology 2012, 153, 1585-92; and Fong et al., J. Ovarian Res. 2009, 2, 12. Models for determining efficacy of treatments for pancreatic cancer are described in Herreros-Villanueva et al., World 1 Gastroenterol. 2012, 18, 1286-1294. Models for determining efficacy of treatments for breast cancer are described, e.g., in Fantozzi, Breast Cancer Res. 2006, 8, 212. Models for determining efficacy of treatments for melanoma are described, e.g., in Damsky et al., Pigment Cell & Melanoma Res. 2010, 23, 853-859. Models for determining efficacy of treatments for lung cancer are described, e.g., in Meuwissen et al., Genes & Development, 2005, 19, 643-664. Models for determining efficacy of treatments for lung cancer are described, e.g., in Kim, Clin. Exp. Otorhinolaryngol. 2009, 2, 55-60; and Sano, Head Neck Oncol. 2009, 1, 32. Models for determining efficacy in B cell lymphomas, such as diffuse large B cell lymphoma (DLBCL), include the PiBCL1 murine model with BALB/c (haplotype H-2d) mice. Illidge et al., Cancer Biother. & Radiopharm. 2000, 15, 571-80. Efficacy of treatments for Non-Hodgkin's lymphoma may be assessed using the 38C13 murine model with C3H/HeN (haplotype 2-Hk) mice or alternatively the 38C13 Her2/neu model. Timmerman et al., Blood 2001, 97, 1370-77; Penichet et al., Cancer Immunolog. Immunother. 2000, 49, 649-662. Efficacy of treatments for chronic lymphocytic leukemia (CLL) may be assessed using the BCL1 model using BALB/c (haplotype H-2d) mice. Dutt et al., Blood 2011, 117, 3230-29.

### Pharmaceutical Compositions and Routes of Administration

In an embodiment, an active pharmaceutical ingredient or combination of active pharmaceutical ingredient, such as any of the foregoing ER antagonists and immunotherapeutic agents, is provided as a pharmaceutically acceptable composition.

In some embodiments, the concentration of each of the active pharmaceutical ingredients provided in the pharmaceutical compositions of the invention, such as any of the foregoing chemotherapeutic regimens, is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of each of the active pharmaceutical ingredients provided in the pharmaceutical compositions of the invention, such as any of the foregoing chemotherapeutic regimens, is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each of the active pharmaceutical ingredients provided in the pharmaceutical compositions of the invention, such as any of the foregoing chemotherapeutic regimens, is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of each of the active pharmaceutical ingredients provided in the pharmaceutical compositions of the invention, such as any of the foregoing chemotherapeutic regimens, is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of each of the active pharmaceutical ingredients provided in the pharmaceutical compositions of the invention, such as any of the foregoing chemotherapeutic regimens, is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of each of the active pharmaceutical ingredients provided in the pharmaceutical compositions of the invention, such as any of the foregoing chemotherapeutic regimens, is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the active pharmaceutical ingredients according to the invention is effective over a wide dosage range. For example, in the treatment of adult humans, dosages independently range from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician. The clinically-established dosages of the foregoing chemotherapeutic regimens may also be used if appropriate.

In an embodiment, the molar ratio of two active pharmaceutical ingredients in the pharmaceutical compositions is in the range from 10:1 to 1:10, from 2.5:1 to 1:2.5, and about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

Described below are non-limiting pharmaceutical compositions and methods for preparing the same.

In some embodiments, the invention provides a pharmaceutical composition for oral administration containing the active pharmaceutical ingredient or combination of active pharmaceutical ingredients, such as the chemotherapeutic regimens described herein, and a pharmaceutical excipient suitable for oral administration.

In some embodiments, the invention provides a solid pharmaceutical composition for oral administration containing: (i) an effective amount of an active pharmaceutical ingredient or combination of active pharmaceutical ingredients, and (ii) a pharmaceutical excipient suitable for oral administration. In selected embodiments, the composition further contains (iii) an effective amount of a third active pharmaceutical ingredient and optionally (iv) an effective amount of a fourth active pharmaceutical ingredient.

In some embodiments, the invention provides a pharmaceutical composition for injection containing an active pharmaceutical ingredient or combination of active pharmaceutical ingredients, such as an active pharmaceutical ingredient in the chemotherapeutic regimens described herein, and a pharmaceutical excipient suitable for injection.

The forms in which the compositions of the invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an active pharmaceutical ingredient or combination of active pharmaceutical ingredients in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical compositions of the chemotherapeutic regimens described herein may also be prepared from compositions described herein and one or more pharmaceutically acceptable excipients suitable for topical, inhalation, sublingual, buccal, rectal, intraosseous, intraocular, intranasal, epidural, or intraspinal administration. Preparations for such pharmaceutical compositions are well-known in the art. See, e.g., Anderson et al., eds., Handbook of Clinical Drug Data, Tenth Edition, McGraw-Hill, 2002; and Pratt and Taylor, eds., Principles of Drug Action, Third Edition, Churchill Livingston, 1990, each of which is incorporated by reference herein in its entirety.

Administration of an active pharmaceutical ingredient or combination of active pharmaceutical ingredients or a pharmaceutical composition thereof can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), via local delivery by catheter or stent or through inhalation. The active pharmaceutical ingredient or combination of active pharmaceutical ingredients can also be administered intrathecally.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. In some embodiments, the kits may be provided for treating ER (−) cancer in a subject in need thereof. In some embodiments, the kits may include an ER antagonist and an immunotherapeutic agent.

In some embodiments, the kits include an active pharmaceutical ingredient or combination of active pharmaceutical ingredients, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the invention provides a kit comprising a composition comprising a therapeutically effective amount of an active pharmaceutical ingredient or combination of active pharmaceutical ingredients or a pharmaceutically acceptable salt, solvate, hydrate, cocrystal, or prodrug thereof. These compositions are typically pharmaceutical compositions. The kit is for co-administration of the active pharmaceutical ingredient or combination of active pharmaceutical ingredients, either simultaneously or separately.

In some embodiments, the invention provides a kit comprising (1) a composition comprising a therapeutically effective amount of an active pharmaceutical ingredient or combination of active pharmaceutical ingredients, and (2) a diagnostic test for determining whether a patient's cancer is a particular subtype of a cancer (e.g., an ER (−) cancer). Any of the disclosed diagnostic methods may be utilized in the kit.

The kits described above are for use in the treatment of the diseases and conditions described herein. In an embodiment, the kits are for use in the treatment of cancer. In some embodiments, the kits are for use in treating solid tumor cancers.

In an embodiment, the kits of the invention are for use in the treatment of cancer. In an embodiment, the kits of the invention are for use in the treatment of a cancer selected from the group consisting of lung cancer, breast cancer, endometrial cancer, skin cancer, ovarian cancer, gastric cancer, colorectal cancer, brain cancer, renal cancer, bladder cancer, ureter cancer, pancreatic cancer, prostate cancer, thyroid cancer, head and neck cancer, liver cancer, lymphoid cancer, and splenic cancer. In an embodiment, the kits of the invention are for use in the treatment of a cancer selected from the group consisting of lung cancer, breast cancer, endometrial cancer, and ovarian cancer.

Dosages and Dosing Regimens

The amounts of the pharmaceutical compositions administered using the methods herein, such as the dosages and/or amounts of chemotherapeutic regimens, will be dependent on the human or mammal being treated, the severity of the disorder or condition, the rate of administration, the disposition of the active pharmaceutical ingredients and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of the pharmaceutical compositions and active pharmaceutical ingredients may be provided in units of mg/kg of body mass or in mg/m² of body surface area.

In some embodiments, a pharmaceutical composition or active pharmaceutical ingredient is administered in a single dose. Such administration may be by injection, e.g., intravenous injection, in order to introduce the active pharmaceutical ingredient quickly. However, other routes, including the oral route, may be used as appropriate. A single dose of a pharmaceutical composition may also be used for treatment of an acute condition.

In some embodiments, a pharmaceutical composition or active pharmaceutical ingredient is administered in multiple doses. In an embodiment, a pharmaceutical composition is administered in multiple doses. Dosing may be once, twice, three times, four times, five times, six times, or more than six times per day. Dosing may be once a month, once every two weeks, once a week, or once every other day. In other embodiments, a pharmaceutical composition is administered about once per day to about 6 times per day. In some embodiments, a pharmaceutical composition is administered once daily, while in other embodiments, a pharmaceutical composition is administered twice daily, and in other embodiments a pharmaceutical composition is administered three times daily.

Administration of the active pharmaceutical ingredients in the methods of the invention may continue as long as necessary. In selected embodiments, a pharmaceutical composition is administered for more than 1, 2, 3, 4, 5, 6, 7, 14, or 28 days. In some embodiments, a pharmaceutical composition is administered for less than 28, 14, 7, 6, 5, 4, 3, 2, or 1 day. In some embodiments, a pharmaceutical composition is administered chronically on an ongoing basis—e.g., for the treatment of chronic effects. In some embodiments, the administration of a pharmaceutical composition continues for less than about 7 days. In yet another embodiment the administration continues for more than about 6, 10, 14, 28 days, two months, six months, or one year. In some cases, continuous dosing is achieved and maintained as long as necessary.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an effective dosage of an active pharmaceutical ingredient disclosed herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an active pharmaceutical ingredient disclosed herein is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an effective dosage of an active pharmaceutical ingredient disclosed herein is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg. In some embodiments, an effective dosage of an active pharmaceutical ingredient disclosed herein is about 25 mg, about 50 mg, about 75 mg, about 100 mg, about 125 mg, about 150 mg, about 175 mg, about 200 mg, about 225 mg, or about 250 mg.

In some embodiments, an active pharmaceutical ingredient is administered at a dosage of 10 to 200 mg BID, including 50, 60, 70, 80, 90, 100, 150, or 200 mg BID. In some embodiments, an active pharmaceutical ingredient is administered at a dosage of 10 to 500 mg BID, including 1, 5, 10, 15, 25, 50, 75, 100, 150, 200, 300, 400, or 500 mg BID.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of the combination of the active pharmaceutical ingredient may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Mice and Cell Lines

Female 5-8 week old wild-type (WT) C57BL/6 and congenic Ly5.1 mice were purchased from the Charles River Frederick facility. Esr1 knockout (ERα KO) were purchased from The Jackson Laboratory. Ovariectomies were performed by Charles River staff at 5 weeks of age. Mice were treated with vehicle (0.1% ethanol) or 10 μM estradiol (USP grade, Sigma) drinking water refreshed every 3-4 days. All mice were maintained in pathogen-free barrier facilities. All experiments were conducted according to the approval of the Wistar Institute Institutional Animal Care and Use Committee.

ID8 cells were retrovirally transduced to express Defb29 and Vegf-α (Conejo-Garcia, et al. “Tumor-infiltrating dendritic cell precursors recruited by a beta-defensin contribute to vaculogenesis under the influence of Vegf-A.” Nat. Med. (2004) 10:950-8). MCF-7, MDA-MB-231, and LLC1 cells were obtained from American Type Culture Collection. Peritoneal tumors were initiated in mice by injecting 3X106 ID8-Defb29/Vegf-α cells intraperitoneal (i.p.). Intraperitoneal cells were harvested from tumorbearing mice by flushing the peritoneal cavity with PBS. Cells were maintained in vitro at 37° C., 5% CO2 by culturing in RPMI+10% FBS or steroid free media (SFR10), which was comprised of phenol red-free RPMI+10% charcoal-stripped FBS. Cells were treated in vitro with vehicle (0.1% DMSO) or varying concentrations of estradiol, fulvestrant, or Methylpiperidino pyrazole (MPP) purchased from Cayman Chemical. Cell line proliferation was determined by MTS assay (Promega), and increased/decreased proliferation relative to vehicle was calculated.

For generating mixed BM chimeras, mononuclear BM cells were collected from adult agematched CD45.1 (congenic) WT or CD45.2 Esr1^{-/-} donor mice, and 1-2×10⁶ cells were mixed in a 1:1 ratio and retro-orbitally injected into lethally irradiated (˜950 rad) adult recipients. Mixed chimeras were analyzed after 7-8 weeks as indicated.

### Human Samples

All patients selected for entry into the study met the following criteria: (i) histologically confirmed pulmonary squamous cell carcinoma (SCC) or adenocarcinoma (AC), (ii) no prior chemotherapy or radiation therapy within two years, and (iii) no other active malignancy. BM cell suspension was obtained from rib fragments that were removed from patients as part of their lung cancer surgery. Informed consent was obtained from all subjects.

### Flow Cytometry

Flow cytometry was performed by staining cells with Zombie Yellow viability dye, blocking with anti-CD16/32 (2.4G2), and staining for 30 min at 4° C. with the following anti-mouse antibodies: CD45 (30-F11), CD45.1 (A20), CD45.2 (104), CD11c (N418), I-A/I-E (M5/114.15.2), CD3 (145-2C11), Ly6G (1A8), Ly6C (HK1.4), gp130 (4H1B35), IL6R (D7715A7), Gr1 (RB6-4C5), CD4 (RM4-5), CD8b (YTS156.7.7), CD44 (IM7), CD69 (H1.2F3), CD11b (M1/70); or anti-human antibodies: CD45 (HI30), CD11c (Bul5), HLA-DR-APC/Cy7 (L243), CD15 (HI98), CD14 (HCD14), CD11b (ICRF44), CD33 (WM53), CD19 (HIB19). Samples were subsequently run using an LSRII and analyzed using FlowJo.

### ELISpot

Dendritic cells (BMDCs) were differentiated by culturing WT mouse bone marrow for 7 days with 20 ng/mL GM-CSF (Peprotech 315-03), refreshed every 3 days. BMDCs were subsequently primed with tumor antigen by pulsing for 24 hours with irradiated (100 Gy+30 minutes UV) ID8-Defb29/Vegf-α cells at a ratio of 10:1. ELISPOT assay was performed by stimulating 1X105 cells obtained from peritoneal wash with 1X104 antigen-primed BMDCs in a 96-well filter plate (Millipore MSIPS4510) coated with IFNγ capture antibody according to manufacturer's guidelines (eBioscience 88-3784-88). Following incubation at 37° C., 5% CO2 or 48 hours, positive spots were developed using Avidin-AP and BCIP-NBT substrate (R&D Systems SEL002).

### Adoptive T-Cell Transfer

Naïve T cells were harvested from spleens of WT or ERα KO mice via RBC lysis followed by magnetic bead negative selection to remove non-T cell B220⁺, CD16/32⁺, CD11b⁺ AND MHC-II⁺ cells and primed for 5 days with BMDCs pulsed with tumor antigen. A total of 1×10⁶ T cells was injected i.p. 7 and 14 days post tumor injection.

### Bone Marrow-Derived MDSC Cultures

Mouse MDSCs were expanded from mouse bone marrow harvested by flushing tibias and femurs with media. Following red blood cell lysis, 2.5×10⁶ cells were cultured in 10 mL of RPMI+10% FBS augmented with recombinant mouse 40 ng/mL GM-CSF⁺40 ng/mL IL6 (Peprotech) and incubated at 37° C., 5% CO₂ 3 or 6 days. Vehicle, estradiol, or MPP treatments were added as described above. For 6 day cultures, cytokines and estrogen treatments were refreshed on day 3. Following incubation, floating and adherent cells were collected, and MMDSCs and G-MDSCs were isolated via MDSC purification kit according to manufacturer's protocol for further analysis. Human MDSCs were expanded from human lung cancer patient bone marrow acquired as single cell suspensions (see above). Briefly, 2×10⁶ cells were cultured in 3 mL of IMDM⁺ 15% FBS supplemented with recombinant human 40 ng/mL GM-CSF⁺ 40 ng/mL IL6 (Peprotech) and treated with Vehicle, 2 μM, or 10 μM MPP (see above) for 4 days. Cells were subsequently harvested and analyzed by flow cytometry.

### MDSC Suppression Assay

Naïve WT T cells were purified from spleens as described and labeled with the proliferation tracker Cell Trace Violet according to manufacturer protocol. T cell proliferation was stimulated by adding anti-CD3/CD28 mouse T-activator beads (Thermo) at a 1:1 T cell to bead ratio according to manufacturer protocol. T cells (2×10⁵) were subsequently co-cultured with MDSC at 1:4, 1:8, or 1:16 MDSC to T cell ratios and incubated for 3 days prior to flow cytometric analysis.

### Western Blot

Cells were lysed in RIPA buffer supplemented with protease inhibitors, phosphatase inhibitors, and Na₃VO₄ (Thermo) according to manufacturer protocol. Protein quantification was determined via BCA assay, and protein was run on TGX 4-15% gradient gels. Following transfer, PVDF membranes were blocked with 5% BSA in TBS+ 0.05% Tween-20. The following primary antibodies were added to membranes, as indicated, and incubated overnight: Jak2 (rabbit clone#D2E12), Stat3 (clone#124H6) and pStat3 (Tyr705)(rabbit clone#D3A7), from Cell Signaling; and ERα (Thermo, clone#TE111.5D11) and beta-actin (Sigma, clone#AC-15).

Following secondary staining with HRP-conjugated anti-mouse or rabbit IgG, membranes were developed using ECL prime (GE Healthcare).

### Quantitative Real Time PCR

Cells were lysed in Trizol buffer and RNA was subsequently purified using RNEasy kit (Qiagen). Reverse transcription was carried out using High-Capacity Reverse Transcription kit (Applied Biosystems). SYBR Green PCR Master Mix (Applied Biosystems) was used with an ABI 7500 Fast Sequence Detection Software (Applied Biosystems). The following primer sequences were used (5′-3′): Stat3 <F: GACTGATGAAGAGCTGGCTGACT (SEQ ID NO:75), R:GGGTCTGAAGTTGAGATTCTGCT>(SEQ ID NO:76); Jak2<F: GTGTCGCCGGCCAATGTTC (SEQ ID NO:77), R: CACAGGCGTAATACCACAAGC (SEQ ID NO:78)>; and Tbp (mRNA normalization)< F:CACCCCCTTGTACCCTTCAC (SEQ ID NO:79), R: CAGTTGTCCGTGGCTCTCTT (SEQ ID NO:80)>.

Expression of human ESR1 was quantified with primers: ESR1 <F:CCACTCAACAGCGTGTCTC (SEQ ID NO:81), and R: GGCAGATTCCATAGCCATAC (SEQ ID NO:82)>, and normalized with primers: GAPDH <F: CCTGCACCACCAACTGCTTA (SEQ ID NO:83), R: AGTGATGGCATGGACTGTGGT (SEQ ID NO:84)>.

Expression of mouse ERα was determined with primers: ERα <F:GTGCAGCACCTTGAAGTCTCT (SEQ ID NO:85), R: TGTTGTAGAGATGCTCCATGCC (SEQ ID NO:86)>.

The foregoing sequences are summarized in Table 4.

TABLE 4

| | |
|---|---|
| Identifier | Sequences |

| | | |
|---|---|---|
| SEQ ID NO: 75 | gactgatgaa gagctggctg act | 23 |
| STAT3 Primer | | |
| forward | | |
| SEQ ID NO: 76 | gggtctgaag ttgagattct gct | 23 |
| STAT3 Primer | | |
| reverse | | |
| SEQ ID NO: 77 | gtgtcgccgg ccaatgttc | 19 |
| JAK2 Primer | | |
| forward | | |
| SEQ ID NO: 78 | cacaggcgta ataccacaag c | 21 |
| JAK2 Primer | | |
| reverse | | |
| SEQ ID NO: 79 | cacccccttg tacccttcac | 20 |
| Tbp Primer | | |
| forward | | |
| SEQ ID NO: 80 | cagttgtccg tggctctctt | 20 |
| Tbp Primer | | |
| reverse | | |
| SEQ ID NO: 81 | ccactcaaca gcgtgtctc | 19 |
| ESR1 Primer | | |
| forward | | |
| SEQ ID NO: 82 | ggcagattcc atagccatac | 20 |
| ESR1 Primer | | |
| reverse | | |
| SEQ ID NO: 83 | cctgcaccac caactgctta | 20 |
| GAPDH Primer | | |
| forward | | |
| SEQ ID NO: 84 | agtgatggca tggactgtgg t | 21 |
| GAPDH Primer | | |
| reverse | | |
| SEQ ID NO: 85 | gtgcagcacc ttgaagtctc t | 21 |
| ERa Primer | | |
| forward | | |
| SEQ ID NO: 86 | tgttgtagag atgctccatg cc | 22 |
| ERa Primer | | |
| reverse | | |

### Example 1—Estrogen Signaling Impairs Protective Immunity Against Ovarian Cancer Independently of Tumor Cell Signaling

The role of estrogens in anti-tumor immunity remains poorly understood. Here it is shown that estrogen signaling accelerates the progression of different estrogen insensitive tumor models by contributing to deregulated myelopoiesis by both driving the mobilization of Myeloid-derived Suppressor Cells (MDSCs) and enhancing their intrinsic immunosuppressive activity in vivo. Differences in tumor growth are dependent on blunted anti-tumor immunity and, correspondingly, disappear in immunodeficient hosts. Mechanistically, estrogen receptor alpha activates the JAK-STAT3 pathway in human and mouse bone marrow myeloid precursors by upregulation of JAK2 and, subsequently, enhancing IL-6-driven overexpression of total STAT3. Therefore, estrogen signaling is a crucial mechanism underlying pathological myelopoiesis in cancer. Our work suggests that new anti-estrogen drugs that have no agonistic effects may have benefits in a wide range of cancers, independently of the expression of estrogen receptors in tumor cells, and may synergize with immunotherapies to significantly extend survival.

Nuclear expression of ERs specifically in neoplastic cells has been identified in human ovarian carcinomas of all histological subtypes, with strong signal in ˜60% of high-grade serous tumors. ERα is the predominant estrogen receptor in at least mouse hematopoietic cells. To define the expression of ERα in human ovarian cancer-infiltrating leukocytes, immunohistochemical analysis is first performed in 54 serous ovarian carcinomas. Positive staining was found in tumor cells in ˜35% of tumors (FIG. 1A, left), along with weaker signal in individual stromal cells (not shown). In addition, a second class of ovarian tumors was identified in which ERα expression was confined to individual cells in the stroma (FIG. 1A, right). To confirm that hematopoietic cells at tumor beds indeed express ERα, cells (CD45⁺) were sorted from 7 different dissociated human ovarian tumors. As shown in FIG. 1B, both tumor-infiltrating (CD11b⁺) myeloid cells and (CD11b⁻) non-myeloid leukocytes express variable levels of ERα. In addition, both myeloid and lymphoid cells sorted from the bone marrow of a cancer patient were also ERα⁺, suggesting that in addition to potentially having tumor cell-intrinsic effects, estrogens may also play wider a role in shaping the ovarian tumor immune-environment. To determine the role of estrogen signaling in tumor-promoting inflammation or anti-tumor immunity, a preclinical model of aggressive ovarian cancer was used in which syngeneic epithelial ovarian tumor cells (ID8-Defb29/Vegf-a) develop intraperitoneal tumors and ascites that recapitulate the inflammatory microenvironment of human ovarian tumors. Importantly, no ERα was detected in these cells, unlike tumor-associated myeloid cells (FIG. 1C). Most importantly, ID8-Defb29/Vegf-α cells fail to respond to estradiol (E2) treatment or ER antagonism in vitro, unlike established estrogen-responsive MCF-7 cells (FIG. 1D). Supporting a tumor cell-independent role of estrogen signaling in malignant progression, oophorectomized (estrogen-depleted) wild-type mice survived significantly longer than non-oophorectomized, aged-matched controls after orthotopic tumor challenge in multiple independent experiments (FIG. 1E). Most importantly, the survival benefit imparted by oophorectomy disappeared in tumor-bearing immunodeficient RAG1-deficient KO mice (FIG. 1F), indicating that an adaptive immune response is required for the protective effects of estrogen depletion.

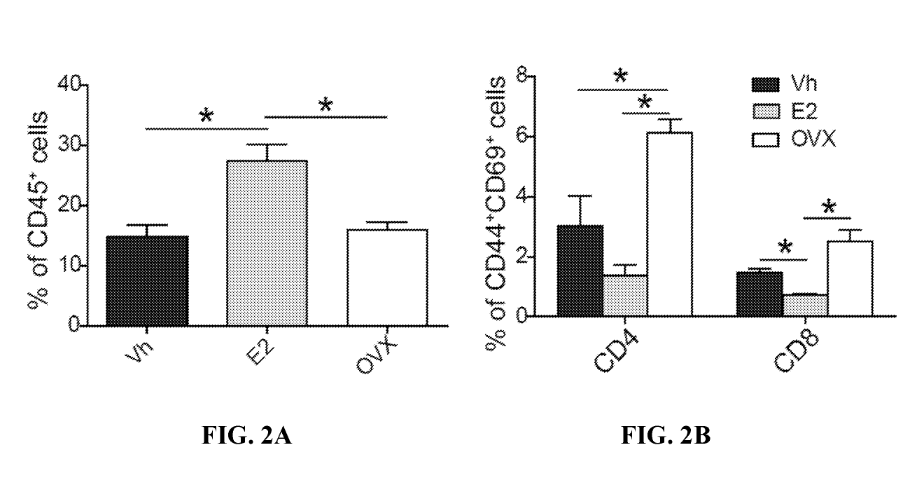

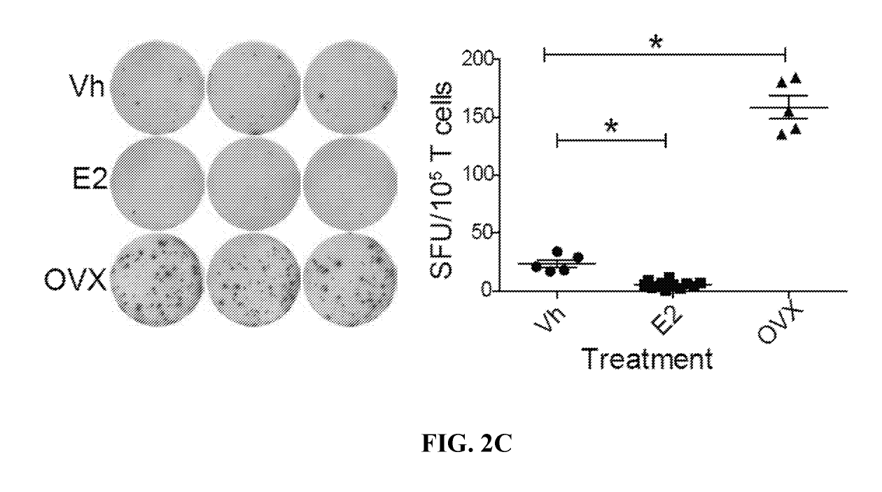

Interestingly, ad libitum estradiol supplementation resulted in augmented inflammation at tumor (peritoneal) beds (FIG. 2A). However, the proportions of antigen experienced (CD44+), recently activated (CD69+) tumor-associated CD4 and CD8 T-cells were significantly higher in oophorectomized tumor-bearing hosts, with corresponding decreases in estradiol-supplemented animals (FIG. 2B). Accordingly, the frequencies of T cells isolated from the peritoneal cavity of oophorectomized tumor-bearing mice producing Interferon (IFN)-γ in response to cognate tumor antigens were significantly higher than those generated by control (non-oophorectomized) mice in conventional ELISpot analysis (FIG. 2C), indicative of superior T cell-dependent anti-tumor immunity. Consistently, tumor-associated T cells from E2-treated mice responded significantly worse than either group (FIG. 2C). Taken together, these results demonstrate that estrogens accelerate ovarian cancer progression, independent of a direct effect on tumor cells, through a mechanism that blunts protective anti-tumor immunity.

### Example 2—ERα Signaling in Hematopoietic Cells Enhances Ovarian Cancer-Induced Myelopoietic Expansion

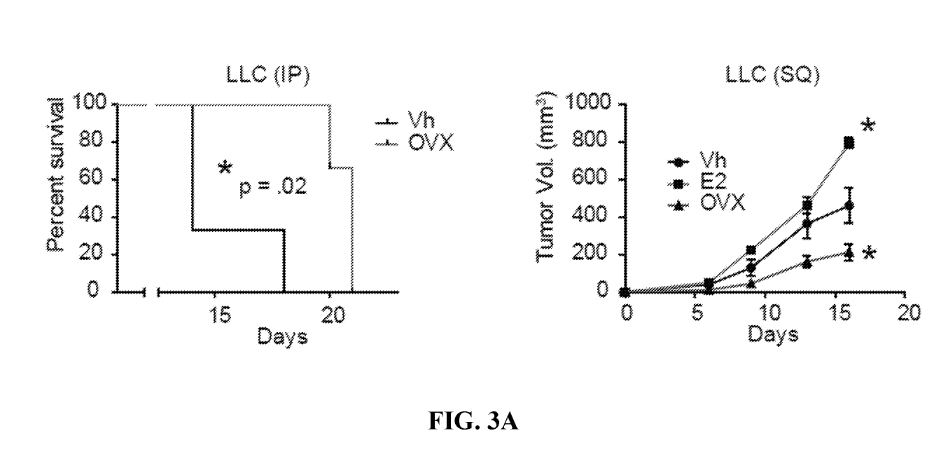

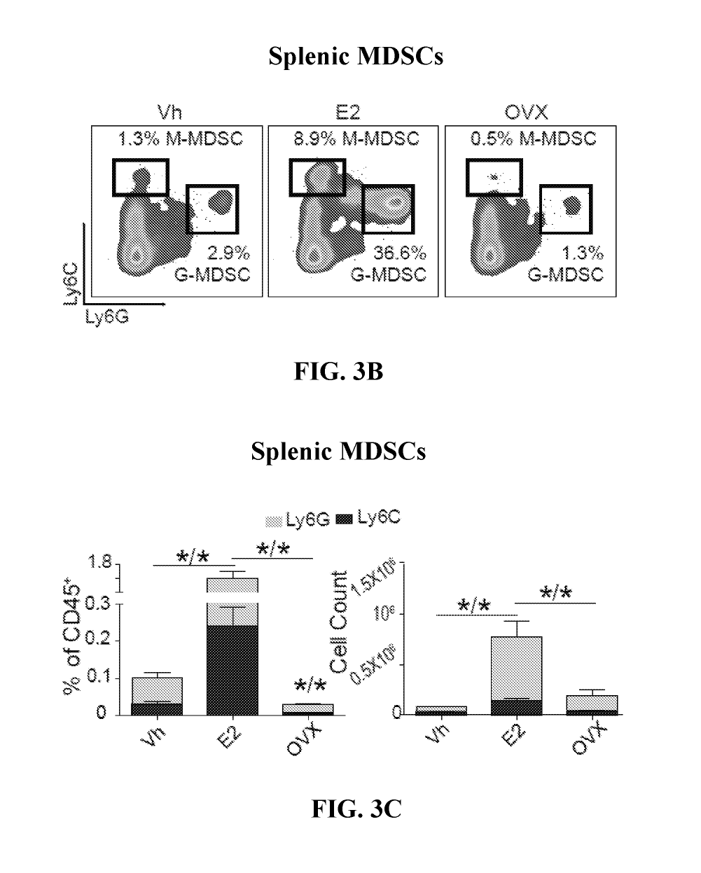

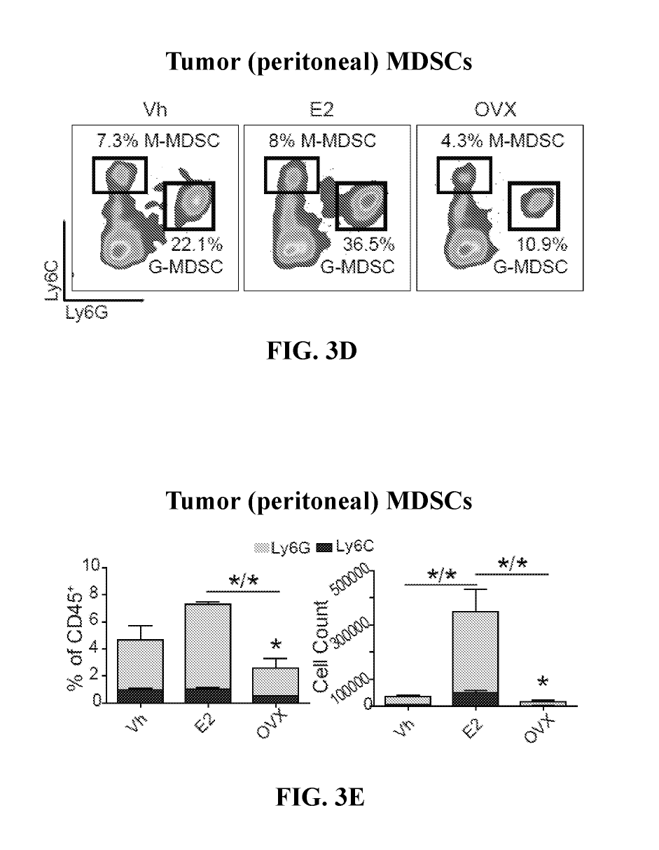

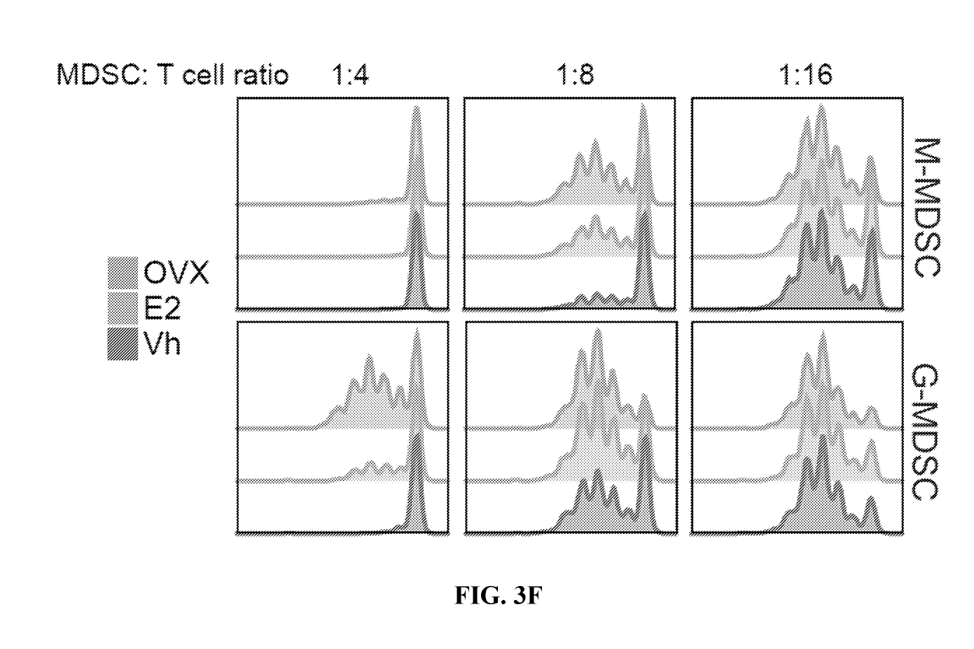

The benefits of estrogen depletion were not restricted to ID8-Defb29/Vegf-α tumors, because the progression of male-derived, estrogen-insensitive (not shown), intraperitoneal Lewis Lung Carcinomas (LLC) was also significantly delayed in oophorectomized mice, while estradiol supplementation accelerated malignant growth, ultimately resulting in decreased survival (FIG. 3A).

To determine the mechanism by which estrogen signaling accelerates malignant progression, differences in the mobilization of immunosuppressive cells were next investigated. Strong estrogen-dependent differences were identified only in the accumulation of myeloid derived suppressor cells (MDSCs), both in the spleen (FIGS. 3B and 3C) and at tumor beds (FIGS. 3D and 3E). Hence, estrogen treatment increased the percentage and total numbers of both Ly6C^{high}Ly6G− myelomonocytic (M-MDSC) and Ly6C+Ly6G+ granulocytic MDSCs (G-MDSC) in tumor-bearing mice, while estrogen depletion through oophorectomy significantly decreased their percentage and total numbers both in the spleen and at tumor beds (FIGS. 3B to 3E).

Estrogens primarily signal through the nuclear receptors ERα and ERβ, the former being expressed in virtually all murine hematopoietic cells. Further supporting that differences in the ovarian cancer immuno-environment are independent of estrogen signaling on tumor cells, ERα expression was identified in MDSCs derived from tumor-derived mice (FIG. 1C). Importantly, myeloid cells sorted from tumor-bearing mice were also highly effective at suppressing T-cell proliferative responses and therefore are true immunosuppressive MDSCs and not merely immature hematopoietic cells (FIG. 3F), supporting their role in estrogen dependent abrogation of anti-tumor immunity. Interestingly, G-MDSCs from E2-depleted (oophorectomized) mice exhibit weaker immunosuppressive potential compared to vehicle or E2-treated mice.

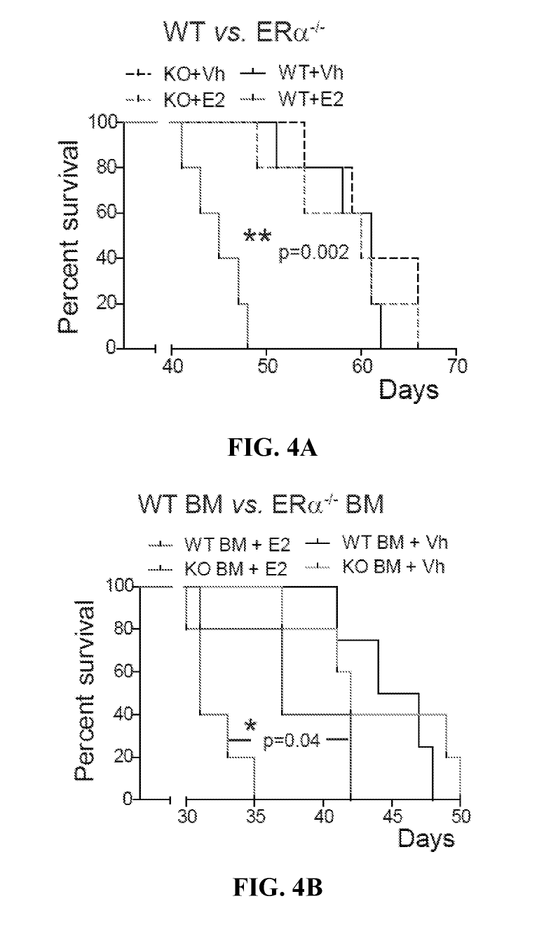

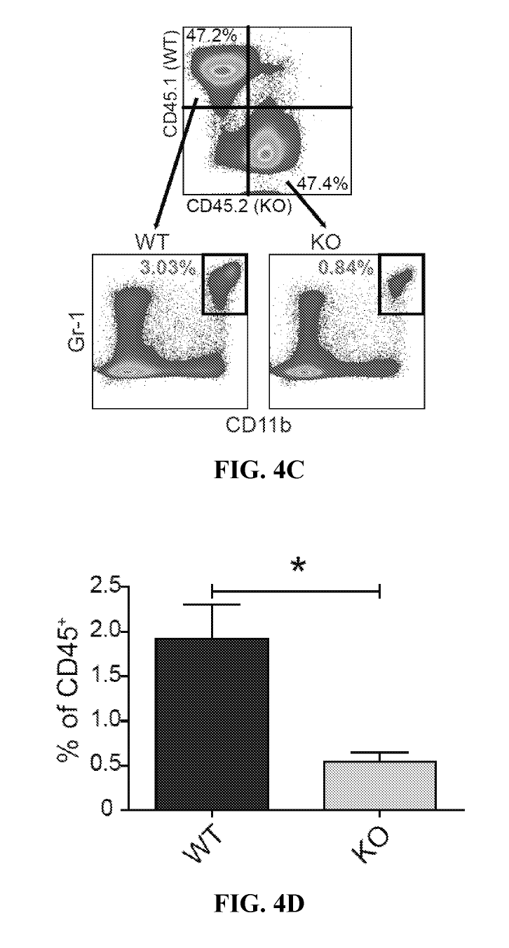

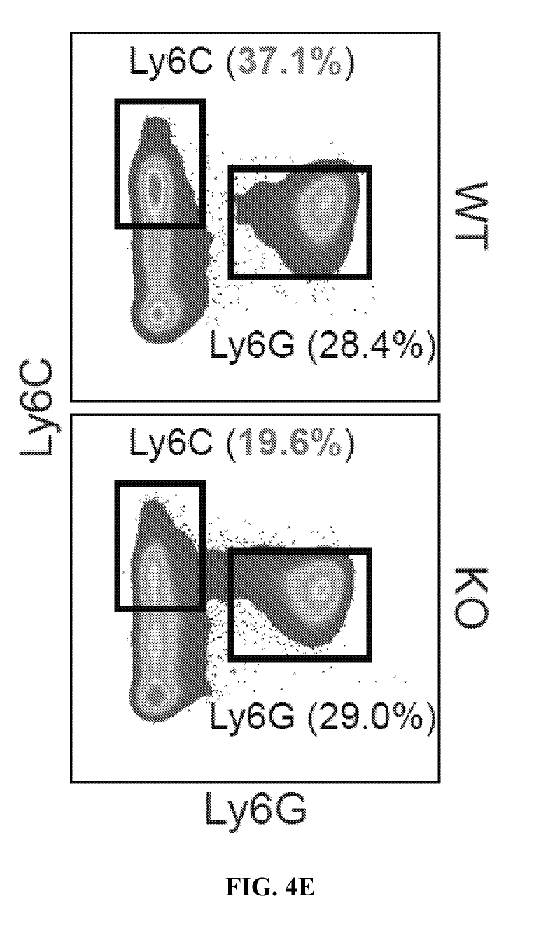

To confirm that ERα signaling is sufficient to mediate accelerated malignant progression, ERα^{-/-} and wild-type control mice were then challenged with orthotopic ID8-Defb29/Vegf-α tumors. As shown in FIG. 4A, estradiol supplementation failed to accelerate tumor progression in ERα KO hosts but again had significant effects in wild-type controls, indicative that estrogen's tumor-promoting responses are attributable to ERα signaling. Most importantly, accelerated tumor growth depends on ERα signaling specifically on hematopoietic cells because in response to E2 treatment, tumors progress significantly faster in lethally irradiated mice reconstituted with wild-type bone marrow, compared to identically treated mice reconstituted with ERα-deficient bone marrow (FIG. 4B). Together, these results indicate that ERα signaling on hematopoietic cells accelerates malignant progression independently of the stimulation of neoplastic cells, through a mechanism that results in the mobilization of (ERα⁺) immunosuppressive MDSCs.

### Example 3—Estrogens Signal Through ERα on Human and Mouse Myeloid Progenitors to Boost the Proliferation of Regulatory Myeloid Cells and Enhance their Immunosuppressive Activity

To rule out that estrogen-dependent myeloid expansion in tumor-bearing mice was the result of subtle differences in tumor burden or inflammation, lethally irradiated mice were reconstituted with a 1:1 mixture of CD45.2⁺ERα^{-/-} and (congenic) CD45.1⁺ERα⁺ bone marrow and challenged them with orthotopic ovarian tumors. As shown in FIGS. 4C and 4D, a significantly higher percentage (3.6-fold) of total (CD11b⁺Gr-1⁺) MDSCs arose from ERα⁺ hematopoietic progenitors, compared to ERα-deficient cells. Because reconstitution of total hematopoietic cells occurred at a similar ratio (FIG. 4C) and MDSCs mobilization took place in the same host under an identical milieu, dissimilar ERα-dependent MDSC accumulation can only be attributed to cell-intrinsic ERα+ signaling on myeloid precursors. Notably, a preferential decrease in the expansion of ERα-deficient M-MDSCs was found, compared to myeloid cells of the granulocytic lineage (FIG. 4E), although the total count of both populations was nevertheless diminished in the absence of ERα signaling (not shown).

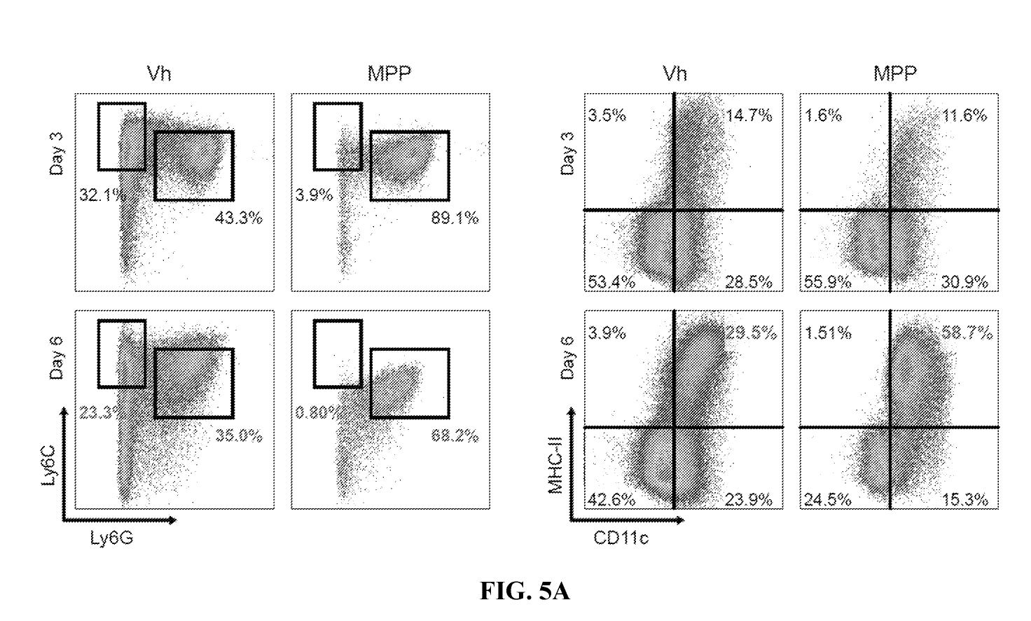

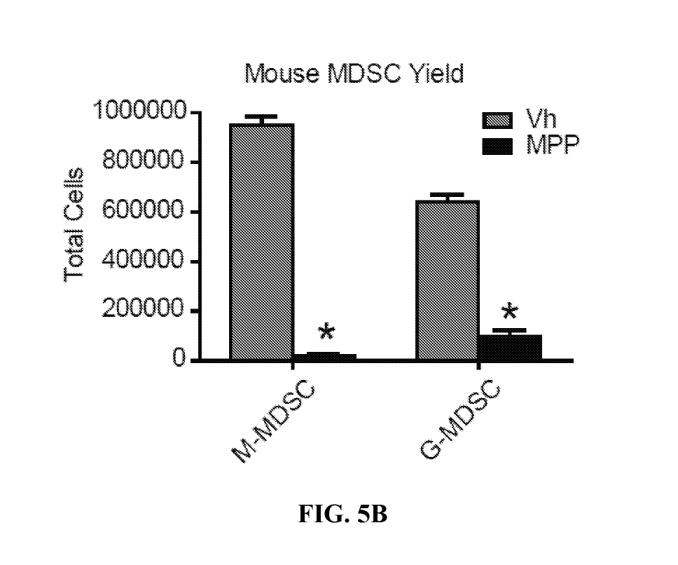

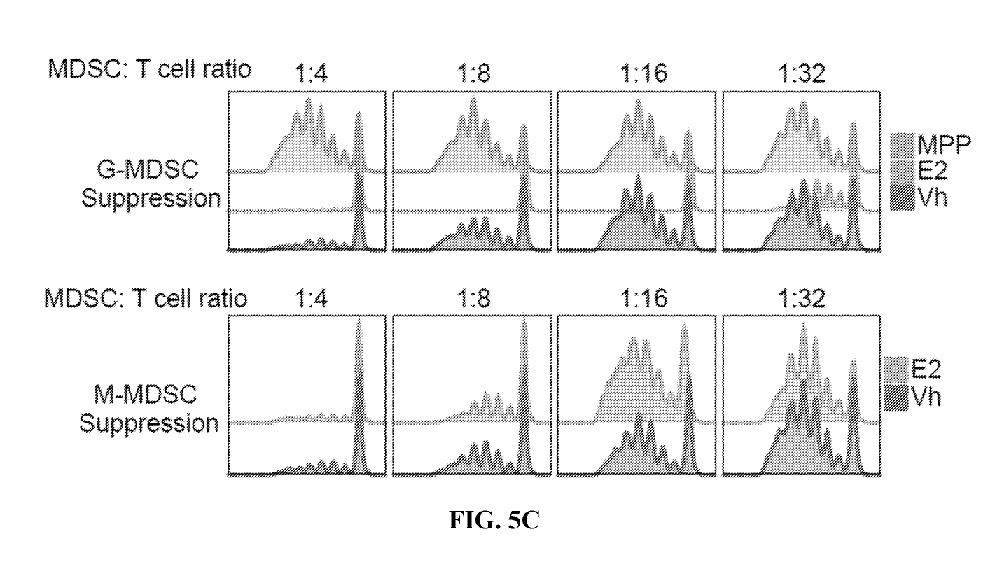

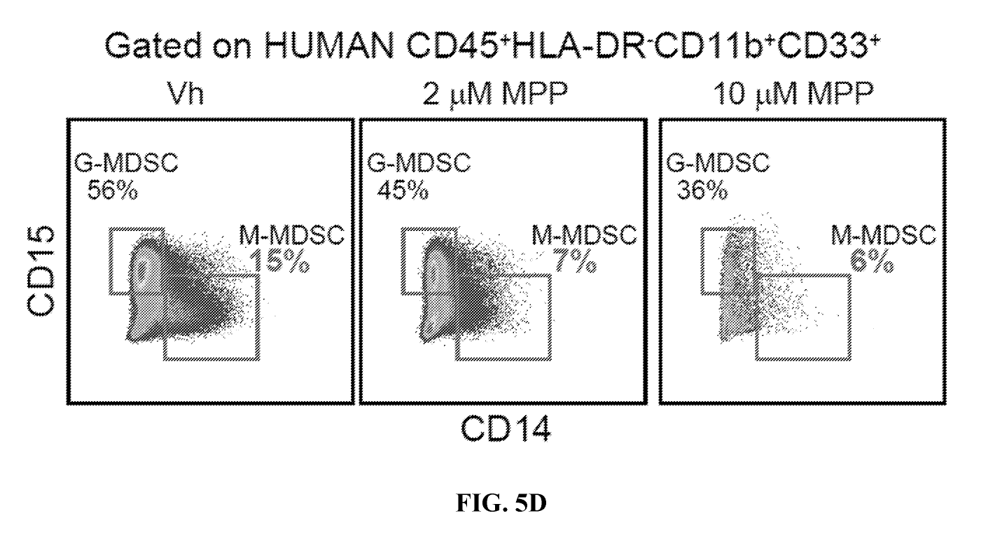

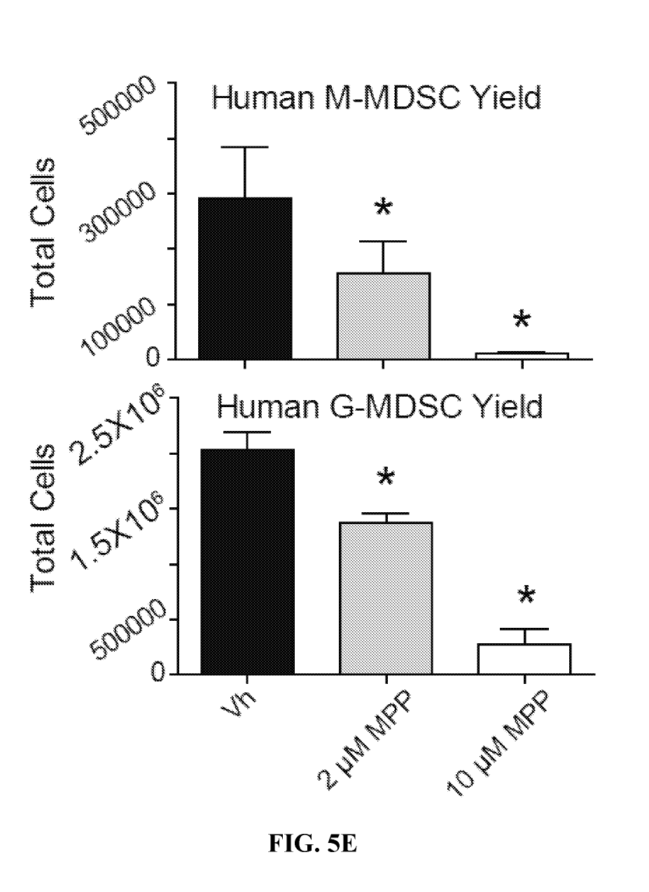

To understand how estrogen signaling promotes MDSC expansion, MDSCs in vitro were next differentiated by treating naïve wild-type (ERα⁺) BM with GM-CSF and IL-6. These inflammatory cytokines induced the generation of immature myeloid cells that express Ly6G and Ly6C similar to MDSCs seen in vivo (FIG. 5A, left).

Normal cell culture media drives estrogen signaling due to the presence of various estrogens in FBS in addition to the estrogenic properties of phenol red. Blocking the estrogen activity of cell culture media with MPP, a selective antagonist of ERα, severely inhibited the expansion of both M-MDSCs and G-MDSCs, with a preferential effect on the former (FIG. 5A, left, and FIG. 5B), similar to in vivo in tumor-bearing mice (FIG. 4E). In addition, the presence ERα antagonists allowed spontaneous differentiation of CD11c⁺MHC-II⁺ dendritic-like cells (FIG. 5A, right). Corresponding to in vivo observations (FIG. 3F), further addition of estradiol resulted in G-MDSCs that were more potently immunosuppressive while abrogation of ERα signaling prevented the acquisition of stronger immunosuppressive activity by G-MDSCs (FIG. 5C, top). In contrast, estradiol did not affect the inhibitory activity of M-MDSCs (FIG. 5C, bottom) suggesting that the role of estrogens in the accumulation of M-MDSCs is to primarily drive their expansion, although the low yields of BMMDSCs obtained in the presence of estrogen antagonists precludes testing their suppressive activity.

To support the relevance of ERα signaling in boosting pathological expansion of MDSCs, bone marrow was procured from 5 different lung cancer patients, and expanded myeloid cells with GM-CSF and IL-6, in the presence of different concentrations of an ERα antagonist (MPP). As shown in FIG. 5D, this system results in reproducible expansion of CD11b⁺CD33⁺CD15^{-/low}CD14⁻MHC-If granulocytes and CD11b⁺CD33⁺CD15⁻CD14⁺MHC-II monocytic cells, corresponding to the human counterparts of granulocytic and monocytic MDSCs. Notably, blockade of ERα signaling resulted in a dramatic dose-dependent reduction in the expansion of both MDSC lineages, both at the level of proportions (FIG. 5D) and, especially, absolute numbers (FIG. 5E). Together, these data show that estrogen signaling through ERα influences myelopoiesis in both mice and humans, ultimately boosting the expansion of MDSCs in response to inflammatory signals; contributing to enhance their immunosuppressive activity; and blocking their differentiation into MHC-II⁺ myeloid cells, overall promoting malignant progression.

### Example 4—Estrogens Signaling Enhances pSTAT3 Activity Through Transcriptional Up-Regulation of Janus Kinase 2 (JAK2) and Increased Total STAT3 Expression in Myeloid Progenitors

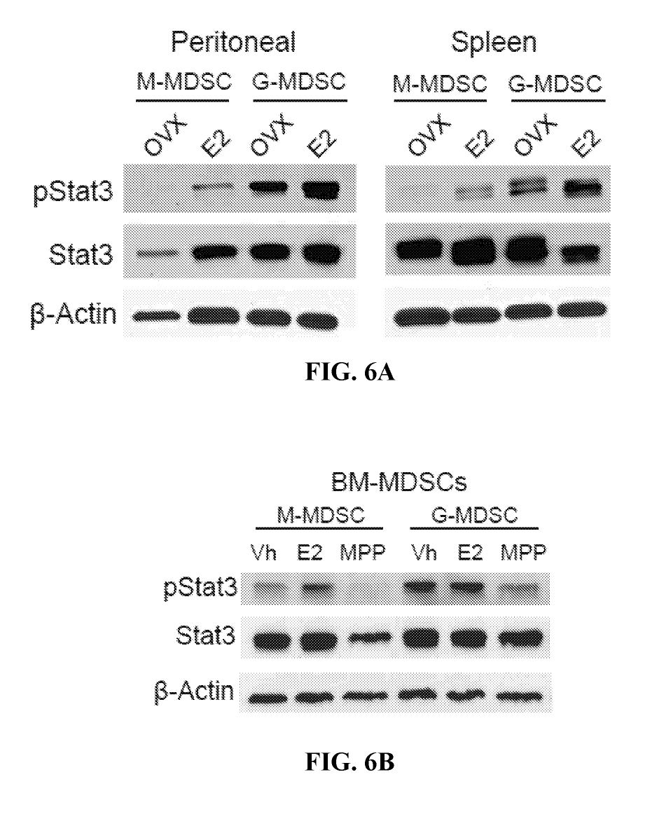

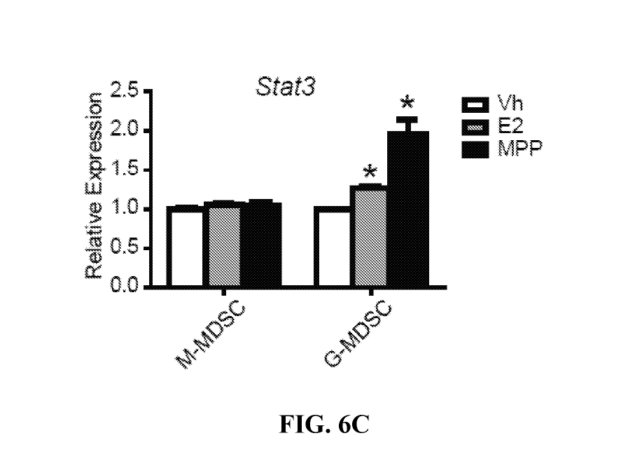

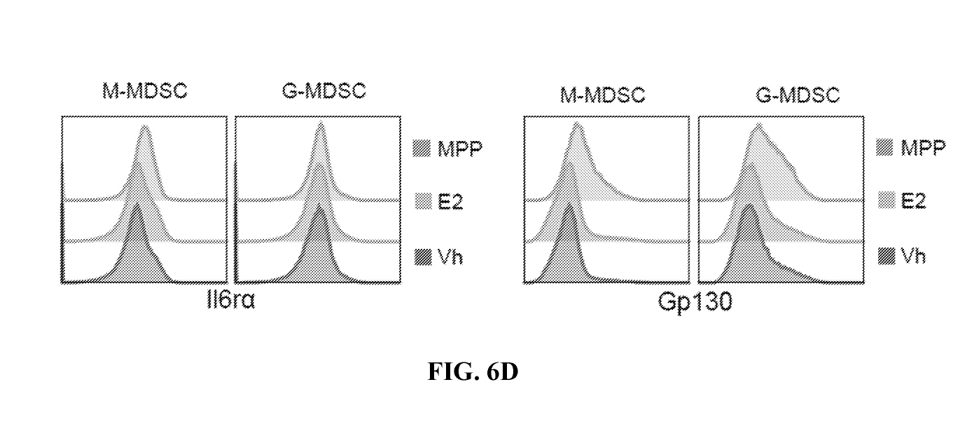

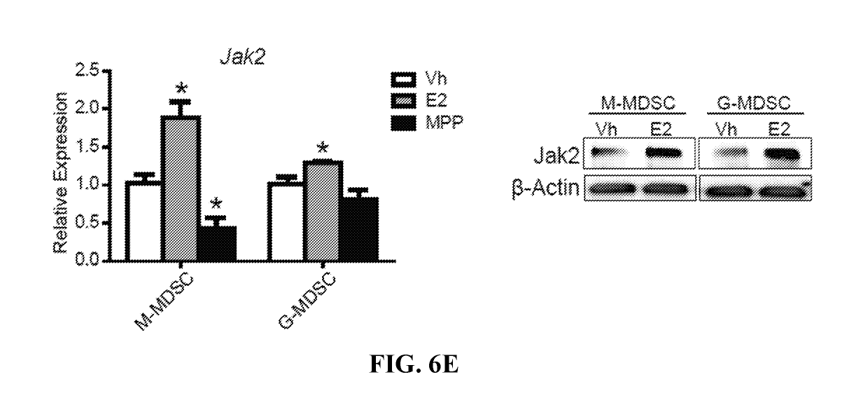

To determine the mechanism by which estrogen signaling promotes MDSC mobilization, the effect of estrogen signaling on STAT3 signaling was focused upon, which plays a major role in regulating myeloid lineage cells and MDSC expansion. As shown in FIG. 6A, levels of pSTAT3Y705 were significantly increased in monocytic and, to a lesser extent, granulocytic MDSCs immunopurified from the spleens of advanced ovarian cancer-bearing mice treated with E2, compared to oophorectomized mice. Accordingly, anti-estrogen treatment of in vitro BMMDSCs cultures inhibited STAT3 signaling resulting in lower phospho-STAT3 in both MMDSCs and G-MDSCs (FIG. 6B), indicating that pSTAT3 signaling is enhanced by estrogen signaling. Finally, E2 supplementation of cell culture media increased phospho-STAT3 levels with more obvious activity on M-MDSCs (FIG. 6B).

Interestingly, total STAT3 was up-regulated in tumor-bearing host-derived M-MDSCs, likely as a direct effect of estrogen-driven enhanced IL-6 signaling. Accordingly, anti-estrogen drugs down-regulated total STAT3 in BM-derived M-MDSC (FIGS. 6A and 6B). However, this difference does not appear to be transcriptionally regulated, as no differences STAT3 transcription was observed in M-MDSCs and statistically significant but only slight differences were observed in G-MDSCs (FIG. 6C).

Because STAT3 activation is triggered by IL-6, which was used for in vitro MDSC expansion, the role of estrogen signaling on IL6R was investigated. Treating BM-MDSCs with E2 or anti-estrogens did not elicit changes in surface expression of the IL6Rα chain (FIG. 6D, left) or gp130 (FIG. 6D, right), suggesting that estrogen signaling could affect downstream mediators. Jak activation was focused upon, which mediates STAT3 phosphorylation, subsequent dimerization, and nuclear translocation following cytokine receptor engagement. As shown in FIG. 6E, left, estrogen supplementation induced transcriptional up-regulation of Jak2 in cytokine-induced bone marrow MDSCs of both lineages, while no detectable expression or changes were identified for other Jak members (not shown). Most importantly, estradiol also induced a reproducible Jak2 up-regulation at the protein level (FIG. 6E, right). Therefore, ERα signaling on myeloid precursors drives MDSC expansion by amplifying IL-6 activity and, subsequently enhancing JAK-STAT3 signaling. This occurs downstream of the receptor through up-regulation of total STAT3 and increased Jak2 activity.

### Example 5—Estrogen Also Impacts Other Components of the Tumor Immune-Environment

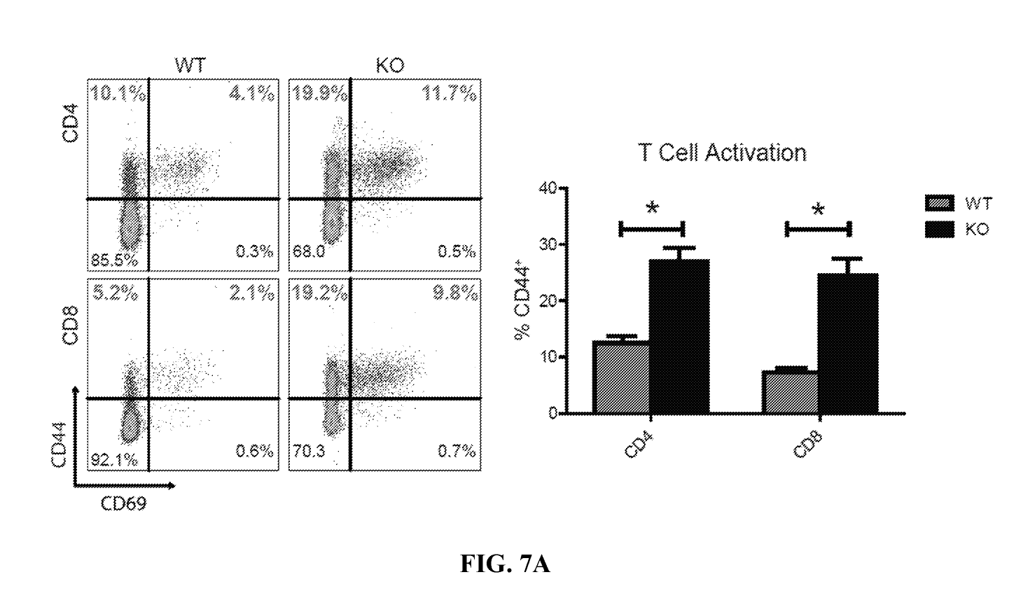

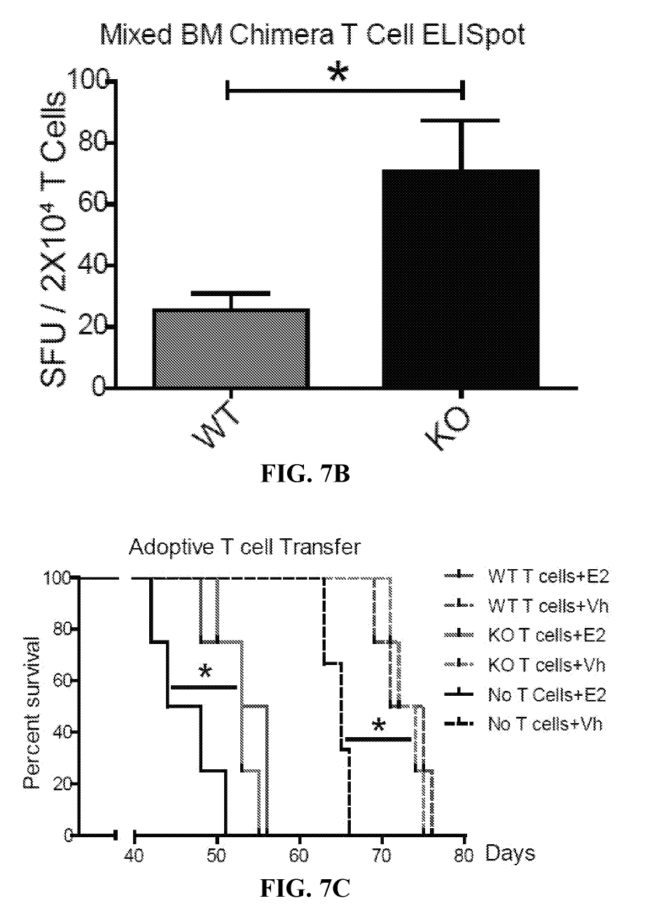

Finally, to rule out that differences in malignant progression due to the direct effect of estrogens on effector T cells, mixed BM chimera experiments were performed in which mice received a 1:1 mixture of ERα^{-/-} and congenic wild-type BM. Compared to ERα^{-/-} T cells, E2-responsive wild-type CD4 and CD8 T cells display a less activated phenotype characterized by lower expression of CD44 (FIG. 7A). Correspondingly, the frequencies of wild-type T cells responding to tumor antigens in IFNγ ELISpot re-challenge assays were lower than those of their counterpart ERα^{-/-} T cells, sorted from the same microenvironment (FIG. 7B).

To determine the relative importance of these differences in direct ERα signaling in T cells, independently of estrogen-dependent MDSC activity, wild-type and ERα^{-/-} T cell splenocytes were identically enriched for tumor-reactive populations by ex vivo priming against tumor lysate-pulsed BMDCs, and then adoptively transferred into ovarian cancerbearing mice. Both wild-type and ERα^{-/-} T cells significantly extended survival; however, there was no difference between wild-type and ERα KO T cells regardless of whether mice were treated with E2 (FIG. 7C). Therefore, while E2 does have a measurable T cell-intrinsic effect, this is not sufficient to drive differences in malignant progression, and, therefore, its effect on immunosuppressive cells, namely MDSCs, is the main driver underlying estrogen-driven tumor acceleration.

A number of patent and non-patent publications may be cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

1. A method of treating an estrogen receptor negative (ER (−)) cancer in a subject with estrogen receptor positive (ER (+)) myeloid-derived suppressor cells (MDSC), comprising administering a therapeutically effective amount of one or more estrogen receptor antagonists to the subject in need thereof.

2. The method of claim 1, wherein the one or more estrogen receptor antagonists are selected from the group consisting of: methylpiperidino pyrazole (MPP), THIQ-40, GDC-0927, H3B-6545, VP-128, (E)-3-(3,5-difluoro-4-((1R,3R)-2-(2-fluoro-2-methyl-propyl)-3-methyl-2,3,4,9-tetrahydro-1H-pyrido[3,4-b]indol-1-yl)phenyl)acrylic acid (AZD9496), (11β,17β)-11-[4-[[5-[(4,4,5,5,5-Pentafluoropentyl)sulfonyl]pentyl]oxy]phenylestra-1,3,5,(10)-triene-3,17-diol (RU 58668), 13-methyl-7-[9-(4,4,5,5,5-pentafluoropentylsulfinyl)nonyl]-7,8,9,11,12,13,14,15,16,17-decahydro-6H-cyclopenta[a]-phenanthrene-3,17-diol (fulvestrant), N-butyl-11-[(7R,8S,9S,13S,14S,17S)-3,17-dihydroxy-13-methyl-6,7,8,9,11,12,14,15,16,17-decahydrocyclopena[a]phenanthren-7-yl]-N-methyl-undecanamide (ICI 164384), (+)-7-pivaloyloxy-3-(4′-pivaloyloxyphenyl)-4-methyl-2-(4″-(2″-piperidinoethoxy)phenyl)-2H-benzopyran (EM-800), and (2S)-3-(4-hydroxyphenyl)-4-methyl-2-[4-[2-(1-piperidyl)ethoxy]phenyl]-2H-chromen-7-ol (EM-652).

3. The method of claim 1, further comprising administering a therapeutically effective amount of an immunotherapeutic agent.

4. The method of claim 3, wherein the immunotherapeutic agent comprises one or more of a CTLA-4 inhibitor, a PD-1 inhibitor, a PD-L1 inhibitor, and an IDO inhibitor.

5. The method of claim 4, wherein the CTLA-4 inhibitor is ipilimumab or tremelimumab.

6. The method of claim 4, wherein the PD-1 inhibitor is selected from the group consisting of nivolumab, pembrolizumab, and pidilizumab.

7. The method of claim 4, wherein the PD-L1 inhibitor is selected from the group consisting of durvalumab, atezolizumab, and avelumab.

8. The method of claim 4, wherein the IDO inhibitor is selected from the group consisting of N-(3-bromo-4-fluorophenyl)-N′-hydroxy-4-((2-(sulfamoylamino)ethyl)amino)-1,2,5-oxadiazole-3-carboximidamide and 1-methyl-D-tryptophan.

9. The method of claim 1, wherein the subject has an elevated level of one or more of estradiol and estrogen.

10. The method of claim 1, wherein the ER (−) cancer is selected from the group consisting of lung cancer, breast cancer, endometrial cancer, skin cancer, ovarian cancer, gastric cancer, colorectal cancer, brain cancer, renal cancer, bladder cancer, ureter cancer, pancreatic cancer, prostate cancer, thyroid cancer, head and neck cancer, liver cancer, lymphoid cancer, and splenic cancer.

11. The method of claim 1, wherein the subject comprises an elevated population of ER (+) MDSCs.

12. A pharmaceutical composition for treating an estrogen receptor negative (ER (−)) cancer in a subject with estrogen receptor positive (ER (+)) myeloid-derived suppressor cells (MDSC), the composition comprising one or more estrogen receptor antagonists and an immunotherapeutic agent in therapeutically effective amounts, and a pharmaceutically acceptable carrier.

13. The composition of claim 12, wherein is the one or more estrogen receptor antagonists are selected from the group consisting of: methylpiperidino pyrazole (MPP), THIQ-40, GDC-0927, H3B-6545, VP-128, (E)-3-(3,5-difluoro-4-((1R,3R)-2-(2-fluoro-2-methyl-propyl)-3-methyl-2,3,4,9-tetrahydro-1H-pyrido[3,4-b]indol-1-yl)phenyl)acrylic acid (AZD9496), (11β,17β)-11-[4-[[5-[(4,4,5,5,5-Pentafluoropentyl)sulfonyl]pentyl]oxy]phenylestra-1,3,5,(10)-triene-3,17-diol (RU 58668), 13-methyl-7-[9-(4,4,5,5,5-pentafluoropentylsulfinyl)nonyl]-7,8,9,11,12,13,14,15,16,17-decahydro-6H-cyclopenta[a]-phenanthrene-3,17-diol (fulvestrant), N-butyl-11-[(7R,8S,9S,13S,14S,17S)-3,17-dihydroxy-13-methyl-6,7,8,9,11,12,14,15,16,17-decahydrocyclopena[a]phenanthren-7-yl]-N-methyl-undecanamide (ICI 164384), (+)-7-pivaloyloxy-3-(4′-pivaloyloxyphenyl)-4-methyl-2-(4″-(2″-piperidinoethoxy)phenyl)-2H-benzopyran (EM-800), and (2S)-3-(4-hydroxyphenyl)-4-methyl-2-[4-[2-(1-piperidyl)ethoxy]phenyl]-2H-chromen-7-ol (EM-652).

14. The composition of claim 12, wherein the immunotherapeutic agent comprises one or more of a CTLA-4 inhibitor, a PD-1 inhibitor, a PD-L1 inhibitor, and an IDO inhibitor.

15. The composition of claim 14, wherein the CTLA-4 inhibitor is ipilimumab or tremelimumab.

16. The composition of claim 14 or 15, wherein the PD-1 inhibitor is selected from the group consisting of nivolumab, pembrolizumab, and pidilizumab.

17. The composition of claim 14, wherein the PD-L1 inhibitor is selected from the group consisting of durvalumab, atezolizumab, and avelumab.

18. The composition of claim 14, wherein the IDO inhibitor is selected from the group consisting of N-(3-bromo-4-fluorophenyl)-N′-hydroxy-4-((2-(sulfamoylamino)ethyl)amino)-1,2,5-oxadiazole-3-carboximidamide and 1-methyl-D-tryptophan.

19. The composition of claim 14, wherein the ER (−) cancer is selected from the group consisting of lung cancer, breast cancer, endometrial cancer, skin cancer, ovarian cancer, gastric cancer, colorectal cancer, brain cancer, renal cancer, bladder cancer, ureter cancer, pancreatic cancer, prostate cancer, thyroid cancer, head and neck cancer, liver cancer, lymphoid cancer, and splenic cancer.

20. The composition of claim 14, wherein the subject comprises an elevated population of ER (+) MDSCs.

21. A method of treating estrogen receptor negative (ER (−)) cancer in a subject comprising the steps of:
(a) obtaining a blood sample from the subject;
(b) analyzing the blood sample for myeloid-derived suppressor cells (MDSCs) and testing the MDSCs with an estrogen receptor-specific protein assay;
(c) determining whether the MDSCs are estrogen receptor positive (ER (+)); and
(d) administering a therapeutically effective amount of one or more estrogen receptor antagonists to the subject in need thereof.

22. The method of claim 21, wherein the one or more estrogen receptor antagonists are selected from the group consisting of methylpiperidino pyrazole (MPP), THIQ-40, GDC-0927, H3B-6545, VP-128, (E)-3-(3,5-difluoro-4-((1R,3R)-2-(2-fluoro-2-methyl-propyl)-3-methyl-2,3,4,9-tetrahydro-1H-pyrido[3,4-b]indol-1-yl)phenyl)acrylic acid (AZD9496), (11β,17β)-11-[4-[[5-[(4,4,5,5,5-Pentafluoropentyl)sulfonyl]pentyl]oxy]phenylestra-1,3,5,(10)-triene-3,17-diol (RU 58668), 13-methyl-7-[9-(4,4,5,5,5-pentafluoropentylsulfinyl)nonyl]-7,8,9,11,12,13,14,15,16,17-decahydro-6H-cyclopenta[a]-phenanthrene-3,17-diol (fulvestrant), N-butyl-11-[(7R,8S,9S,13S,14S,17S)-3,17-dihydroxy-13-methyl-6,7,8,9,11,12,14,15,16,17-decahydrocyclopena[a]phenanthren-7-yl]-N-methyl-undecanamide (ICI 164384), (+)-7-pivaloyloxy-3-(4′-pivaloyloxyphenyl)-4-methyl-2-(4″-(2″-piperidinoethoxy)phenyl)-2H-benzopyran (EM-800), and (2S)-3-(4-hydroxyphenyl)-4-methyl-2-[4-[2-(1-piperidyl)ethoxy]phenyl]-2H-chromen-7-ol (EM-652).

23. The method of claim 21, further comprising administering a therapeutically effective amount of an immunotherapeutic agent.

24. The method of claim 23, wherein the immunotherapeutic agent comprises one or more of a CTLA-4 inhibitor, a PD-1 inhibitor, a PD-L1 inhibitor, and an IDO inhibitor.

25. The method of claim 24, wherein the CTLA-4 inhibitor is ipilimumab or tremelimumab.

26. The method of claim 24, wherein the PD-1 inhibitor is selected from the group consisting of nivolumab, pembrolizumab, and pidilizumab.

27. The method of claim 24, wherein the PD-L1 inhibitor is selected from the group consisting of durvalumab, atezolizumab, and avelumab.

28. The method of claim 24, wherein the IDO inhibitor is selected from the group consisting of N-(3-bromo-4-fluorophenyl)-N′-hydroxy-4-((2-(sulfamoylamino)ethyl)amino)-1,2,5-oxadiazole-3-carboximidamide and 1-methyl-D-tryptophan.

29. The method of claim 21, wherein the subject has an elevated level of one or more of estradiol and estrogen.

30. The method of claim 21, wherein the ER (−) cancer is selected from the group consisting of lung cancer, breast cancer, endometrial cancer, skin cancer, ovarian cancer, gastric cancer, colorectal cancer, brain cancer, renal cancer, bladder cancer, ureter cancer, pancreatic cancer, prostate cancer, thyroid cancer, head and neck cancer, liver cancer, lymphoid cancer, and splenic cancer.


SEQUENCE LISTING (86 sequences)

SEQ ID NO: 1 - protein, 448 aa, artificial sequence
  ipilimumab heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, artificial sequence
  ipilimumab light chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 118 aa, artificial sequence
  ipilimumab variable heavy chain
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 4 - protein, 108 aa, artificial sequence
  ipilimumab variable light chain
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 5 - protein, 5 aa, artificial protein
  ipilimumab variable heavy chain CDR1
SYTMH

SEQ ID NO: 6 - protein, 17 aa, artificial protein
  ipilimumab variable heavy chain CDR2
FISYDGNNKYYADSVKG

SEQ ID NO: 7 - protein, 9 aa, artificial sequence
  ipilimumab variable heavy chain CDR3
TGWLGPFDY

SEQ ID NO: 8 - protein, 12 aa, artificial protein
  ipilimumab variable heavy chain CDR1
RASQSVGSSYLA

SEQ ID NO: 9 - protein, 7 aa, artificial sequence
  ipilimumab variable heavy chain CDR2
GAFSRAT

SEQ ID NO: 10 - protein, 9 aa, artificial sequence
  ipilimumab variable heavy chain CDR3
QQYGSSPWT

SEQ ID NO: 11 - protein, 451 aa, artificial sequence
  tremelimumab heavy chain
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, artificial sequence
  tremelimumab light chain
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 125 aa, artificial sequence
  tremelimumab variable heavy chain
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSS

SEQ ID NO: 14 - protein, 107 aa, artificial sequence
  tremelimumab variable light chain
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 15 - protein, 10 aa, artificial sequence
  tremelimumab variable heavy chain CDR1
GFTFSSYGMH

SEQ ID NO: 16 - protein, 15 aa, artificial sequence
  tremelimumab variable heavy chain CDR2
VIWYDGSNKYYADSV

SEQ ID NO: 17 - protein, 16 aa, artificial sequence
  tremelimumab variable heavy chain CDR3
DPRGATLYYYYYGMDV

SEQ ID NO: 18 - protein, 11 aa, artificial sequence
  tremelimumab variable light chain CDR1
RASQSINSYLD

SEQ ID NO: 19 - protein, 7 aa, artificial sequence
  tremelimumab variable light chain CDR2
AASSLQS

SEQ ID NO: 20 - protein, 9 aa, artificial sequence
  tremelimumab variable light chain CDR3.       Position 3 may alternatively be T or S, position 4 may be F or L,       position 5 may be T or S, and position 7 may be H, S, or T.
QQYYSTPFT

SEQ ID NO: 21 - protein, 440 aa, artificial sequence
  nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 214 aa, artificial sequence
  nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 113 aa, artificial sequence [sequence omitted]

SEQ ID NO: 24 - protein, 107 aa, artificial sequence [sequence omitted]

SEQ ID NO: 25 - protein, 5 aa, artificial sequence
  nivolumab heavy chain CDR1
NSGMH

SEQ ID NO: 26 - protein, 17 aa, artificial sequence
  nivolumab heavy chain CDR2
VIWYDGSKRYYADSVKG

SEQ ID NO: 27 - protein, 4 aa, artificial sequence
  nivolumab heavy chain CDR3
NDDY

SEQ ID NO: 28 - protein, 11 aa, artificial sequence
  nivolumab light chain CDR1
RASQSVSSYLA

SEQ ID NO: 29 - protein, 7 aa, artificial sequence
  nivolumab light chain CDR2
DASNRAT

SEQ ID NO: 30 - protein, 9 aa, artificial sequence
  nivolumab light chain CDR3
QQSSNWPRT

SEQ ID NO: 31 - protein, 447 aa, artificial sequence
  pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 218 aa, artificial sequence
  pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 120 aa, artificial sequence [sequence omitted]

SEQ ID NO: 34 - protein, 111 aa, artificial sequence [sequence omitted]

SEQ ID NO: 35 - protein, 5 aa, artificial sequence
  pembrolizumab heavy chain CDR1
NYYMY

SEQ ID NO: 36 - protein, 16 aa, artificial sequence
  pembrolizumab heavy chain CDR2
GINPSNGGTNFNEKFK

SEQ ID NO: 37 - protein, 11 aa, artificial sequence
  pembrolizumab heavy chain CDR3
RDYRFDMGFDY

SEQ ID NO: 38 - protein, 15 aa, artificial sequence
  pembrolizumab light chain CDR1
RASKGVSTSGYSYLH

SEQ ID NO: 39 - protein, 7 aa, artificial sequence
  pembrolizumab light chain CDR2
LASYLES

SEQ ID NO: 40 - protein, 9 aa, artificial sequence
  pembrolizumab light chain CDR3
QHSRDLPLT

SEQ ID NO: 41 - protein, 447 aa, artificial sequence
  pidilizumab heavy chain
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 213 aa, artificial sequence
  pidilizumab light chain
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 117 aa, artificial sequence
  pidilizumab variable heavy chain
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 44 - protein, 106 aa, artificial sequence
  pidilizumab variable light chain
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 45 - protein, 451 aa, artificial sequence
  durvalumab (MEDI4736) heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 265 aa, artificial sequence
  durvalumab (MEDI4736) light chain
EVQLVESGGGLVQPGGSLRLSCAASGFTFSRYWMSWVRQAPGKGLEWVANEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 121 aa, artificial sequence
  durvalumab variable heavy chain
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 48 - protein, 108 aa, artificial sequence
  durvalumab variable light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 49 - protein, 5 aa, artificial sequence
  durvalumab heavy chain CDR1
RYWMS

SEQ ID NO: 50 - protein, 17 aa, artificial sequence
  durvalumab heavy chain CDR2
NIKQDGSEKYYVDSVKG

SEQ ID NO: 51 - protein, 12 aa, artificial sequence
  durvalumab heavy chain CDR3
EGGWFGELAFDY

SEQ ID NO: 52 - protein, 12 aa, artificial sequence
  durvalumab light chain CDR1
RASQRVSSSYLA

SEQ ID NO: 53 - protein, 7 aa, artificial sequence
  durvalumab light chain CDR2
DASSRAT

SEQ ID NO: 54 - protein, 9 aa, artificial sequence
  durvalumab light chain CDR3
QQYGSLPWT

SEQ ID NO: 55 - protein, 448 aa, artificial sequence
  atezolizumab (MPDL3280A) heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, artificial sequence
  atezolizumab (MPDL3280A) light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 118 aa, artificial sequence
  atezolizumab variable heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 58 - protein, 108 aa, artificial sequence
  atezolizumab variable light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 59 - protein, 10 aa, artificial sequence
  atezolizumab heavy chain CDR1
  MISC_FEATURE (6)..(6): Xaa can be any naturally occurring amino acid
GFTFSXSWIH

SEQ ID NO: 60 - protein, 18 aa, artificial sequence
  atezolizumab heavy chain CDR2
  MISC_FEATURE (4)..(4): Xaa can be any naturally occurring amino acid
  MISC_FEATURE (10)..(10): Xaa can be any naturally occurring amino acid
AWIXPYGGSXYYADSVKG

SEQ ID NO: 61 - protein, 9 aa, artificial sequence
  atezolizumab heavy chain CDR3
RHWPGGFDY

SEQ ID NO: 62 - protein, 11 aa, artificial sequence
  atezolizumab light chain CDR1
  MISC_FEATURE (5)..(7): Xaa can be any naturally occurring amino acid
  MISC_FEATURE (9)..(10): Xaa can be any naturally occurring amino acid
RASQXXXTXXA

SEQ ID NO: 63 - protein, 7 aa, artificial sequence
  atezolizumab light chain CDR2
  MISC_FEATURE (4)..(4): Xaa can be any naturally occurring amino acid
  MISC_FEATURE (6)..(6): Xaa can be any naturally occurring amino acid
SASXLXS

SEQ ID NO: 64 - protein, 9 aa, artificial sequence
  atezolizumab light chain CDR3
  MISC_FEATURE (3)..(6): Xaa can be any naturally occurring amino acid
  MISC_FEATURE (8)..(8): Xaa can be any naturally occurring amino acid
QQXXXXPXT

SEQ ID NO: 65 - protein, 450 aa, artificial sequence
  avelumab (MSB0010718C) heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 216 aa, artificial sequence
  avelumab (MSB0010718C) light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 67 - protein, 120 aa, artificial sequence
  avelumab variable heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 68 - protein, 110 aa, artificial sequence
  avelumab variable light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 69 - protein, 5 aa, artificial sequence
  avelumab heavy chain CDR1
SYIMM

SEQ ID NO: 70 - protein, 17 aa, artificial sequence
  avelumab heavy chain CDR2
SIYPSGGITFYADTVKG

SEQ ID NO: 71 - protein, 11 aa, artificial sequence
  avelumab heavy chain CDR3
IKLGTVTTVDY

SEQ ID NO: 72 - protein, 14 aa, artificial sequence
  avelumab light chain CDR1
TGTSSDVGGYNYVS

SEQ ID NO: 73 - protein, 7 aa, artificial sequence
  avelumab light chain CDR2
DVSNRPS

SEQ ID NO: 74 - protein, 10 aa, artificial sequence
  avelumab light chain CDR3
SSYTSSSTRV

SEQ ID NO: 75 - DNA, 23 bp, artificial sequence
  STAT3 Primer forward
gactgatgaagagctggctgact

SEQ ID NO: 76 - DNA, 23 bp, artificial sequence
  STAT3 Primer reverse
gggtctgaagttgagattctgct

SEQ ID NO: 77 - DNA, 19 bp, artificial sequence
  JAK2 Primer forward
gtgtcgccggccaatgttc

SEQ ID NO: 78 - DNA, 21 bp, artificial sequence
  JAK2 Primer reverse
cacaggcgtaataccacaagc

SEQ ID NO: 79 - DNA, 20 bp, artificial sequence
  Tbp Primer forward
cacccccttgtacccttcac

SEQ ID NO: 80 - DNA, 20 bp, artificial sequence
  Tbp Primer reverse
cagttgtccgtggctctctt

SEQ ID NO: 81 - DNA, 19 bp, artificial sequence
  ESR1 Primer forward
ccactcaacagcgtgtctc

SEQ ID NO: 82 - DNA, 20 bp, artificial sequence
  ESR1 Primer reverse
ggcagattccatagccatac

SEQ ID NO: 83 - DNA, 20 bp, artificial sequence
  GAPDH Primer forward
cctgcaccaccaactgctta

SEQ ID NO: 84 - DNA, 21 bp, artificial sequence
  GAPDH Primer reverse
agtgatggcatggactgtggt

SEQ ID NO: 85 - DNA, 21 bp, artificial sequence
  ER-alpha Primer forward
gtgcagcaccttgaagtctct

SEQ ID NO: 86 - DNA, 22 bp, artificial sequence
  ER-alpha Primer reverse
tgttgtagagatgctccatgcc